(19) United States
(12) Patent Application Publication
(10) Pub. No.: US 2006/0083776 A1
(43) Pub. Date: Apr. 20, 2006

(54) Preparations for topical application and methods of delivering an active agent to a substrate

(76) Inventors: Richard R. Bott, Burlingame, CA (US); Kurt F. Brandstadt, Midland, MI (US); Csilla Kollar, Midland, MI (US); Thomas H. Lane, Midland, MI (US); Donald T. Liles, Midland, MI (US); Mae Saldjeno, Daly City, CA (US); Xavier J. Thomas, Foix (FR); Kevin P. Murphy, Midland, MI (US); Gerald K. Schalau II, Freeland, MI (US)

Correspondence Address: DINSMORE & SHOHL LLP;One Dayton Centre, Suite 1300, One South Main Street, Dayton, OH, 45402-2023, US

(21) Appl. No.: 11/248,017

(22) Filed: Oct. 11, 2005

Related U.S. Application Data
(63) Continuation-in-part of application No. PCT/US04/35686, filed on Oct. 27, 2004.
(60) Provisional application No. 60/514,709, filed on Oct. 27, 2003.

(51) Int. Cl. A61L 15/00

(52) U.S. Cl. 424/445

(57) ABSTRACT

A multi-layer dressing and controlled-release composition for topical application to a substrate include an emulsion and an active agent incorporated into the emulsion. The active agent includes a protein. A method of delivering the active agent to the substrate provides the emulsion and incorporates the active agent into the emulsion for delivery of the active agent to the substrate. Multi-layer dressings and methods particularly suited for management of wound exudate and effective debridement of wound eschar.

### RELATED APPLICATIONS

This application is a continuation-in-part of U.S. Application No. PCT/US2004/035686 filed Oct. 27, 2004, which claims the benefit of U.S. Provisional Application No. 60/514,709, which was filed Oct. 27, 2003.

### STATEMENT OF COOPERATIVE RESEARCH AGREEMENT

The present invention, as defined by the claims herein, was made by parties to a Joint Research Agreement (“Agreement”) between Genencorp International, Inc. and The Dow Corning Corporation, as a result of activities undertaken within the scope of that Agreement. The Agreement was in effect prior to the date of the invention.

### BACKGROUND OF THE INVENTION

1. Field of the Invention

The subject invention generally relates to a multi-layer dressing and a controlled-release composition for topical application to a substrate. The subject invention also generally relates to methods of delivering an active agent to the substrate and to methods of wound exudate management and debridement of wound eschar. The dressing, the controlled-release composition, and the methods of this invention relate are used for topical application to skin and, more particularly, to controlled release dressings comprising emulsions of protein-containing active agents and silicone that, when applied to the skin for therapeutic purposes, provide controlled-release of the active agents from the dressing.

2. Description of the Related Art

Silicones are compounds based on alkylsiloxane or organosiloxane chemistry and include polydimethylsiloxane materials that have been used as excipients and process aids in pharmaceutical applications. Some of these materials have attained the status of pharmacopoeial compounds. Known in the art is the use of such silicone compounds in controlled transdermal drug delivery systems. New long lasting drug delivery applications, including implant, insert, mucoadhesive, and transdermal forms, draw on the unique and intrinsic properties of silicone. Transdermal delivery systems allow controlled-release of active molecules with biologically appropriate kinetics to a targeted area, and prevent the adverse effects, such as peak dosages, low compliance, and drug degradation, commonly observed with traditional oral and parenteral medication.

Transdermal drug delivery systems typically consist of drug containing adhesive patches, which adhere to intact skin up to 7 days. The patch design controls the release of the active agent, which is then transported through the skin and into the organism by the circulatory system for a systemic activity. Using the skin as an entry point, the transdermal forms, which consist of an adhesive plaster or a film-forming and substantive material (e.g., cream or gel), are used for local treatment (muscle or skin disease). Transdermal drug delivery systems have not been incorporated into topical dressing applications such as wound dressings and ointments, wherein a biochemical agent dispersed within a silicone matrix is released onto skin or a wound to accelerate healing.

Specifically, with respect to wound care, non-invasive, rapid debridement of eschar in wounds, chronic wounds in particular, represent an unmet need in wound care. Effective treatment of pressure ulcers such as bed sores, venous leg ulcers and diabetic foot ulcers are of particular concern in our aging population. Non-invasive, easily employed, and effective methods of chronic wound care in a nursing home setting where one health care practitioner is responsible for many patients simultaneously represents a significant and urgent unmet need.

Accordingly, the need remains in the relevant art for compositions, dressing preparations and methods that take advantage of the beneficial properties of silicone to provide controlled release of active agents, and that promote healing of wounds, including chronic wounds.

### SUMMARY OF THE INVENTION AND ADVANTAGES

Multi-layer dressing, controlled-release composition, and method embodiments are disclosed. These elements of the present invention are used for topical application to a substrate. Methods relate to delivery of an active to a substrate, and to management of wound exudates and debridement of wound eschar.

In one embodiment the dressing includes a controlled-release layer and an adhesive layer. The controlled-release layer is formed from the controlled-release composition. The controlled-release composition, more specifically, includes an oil-in-water or water-in-oil emulsion, and the active agent. The active agent is incorporated into the emulsion and comprises a protein. The adhesive layer is disposed adjacent the controlled-release layer and serves to adhere the dressing to the substrate, and/or to adhere the controlled release layer to additional layers, such as a backing layer or occlusive layer. The adhesive layer comprises a silicone adhesive, a non-silicone adhesive, or combinations thereof.

Another embodiment of the present inventive dressings provides a multi-layer dressing comprising: (A) a controlled-release layer formed from a controlled-release composition comprising: (i) an oil-in-water or water-in-oil emulsion, and (ii) an active agent incorporated into said emulsion and comprising a protein; and (B) an adhesive layer disposed adjacent said controlled-release layer; (C) an occlusive layer disposed adjacent said adhesive layer and away from said controlled release layer; (E) an absorbent layer disposed opposite the occlusive layer from the controlled release layer; and, (F) optionally, at least one additional layer selected from the group of a backing layer, a cushioning layer, a second absorbent layer, a second adhesive layer, a porous adhesive layer, and combinations thereof. In a specific embodiment, the additional layer comprises a porous adhesive layer disposed adjacent the absorbent layer and between the absorbent layer and the occlusive layer. In more specific embodiments, the porous adhesive layer comprises a silicone mesh.

One embodiment of the present invention is directed to methods of managing wound exudates and/or achieving noninvasive debridement of wound eschar. In some embodiments the effective management of wound exudates and achievement of noninvasive wound debridement occur simultaneously. The methods comprise application of the inventive dressings to wounds. The methods are particularly effective for the treatment of chronic wounds.

### BRIEF DESCRIPTION OF THE SEVERAL VIEWS OF THE DRAWINGS

The following detailed description of the preferred embodiments of the present invention can be best understood when read in conjunction with the following drawings in which:

FIG. 1A is a cross-sectional side view of a multi-layer dressing according to the present invention;

FIG. 1B is a cross-sectional side view of the dressing of FIG. 1A with a peel-off backing layer removed;

FIG. 1C is a cross-sectional side view of another embodiment of the dressing including additional layers;

FIG. 1D is a cross-sectional side view of yet a further embodiment of the dressing with a ring of adhesive for the adhesive layer;

FIG. 2 is a graph illustrating % Protease B enzyme released from PSA/PVA formulations;

FIG. 3 is a graph illustrating mg of Enzyme released from varying levels of LG12-containing PSA I PVA formulation;

FIG. 4 is a graph illustrating % Protease B enzyme released from 2220 formulations;

FIG. 5 is a graph illustrating % Protease B enzyme released from 9090 formulations;

FIG. 6 is a graph illustrating the effect of glycerin on the % Protease B enzyme released from PSA/PVA formulations;

FIG. 7 is a graph illustrating the effect of processing on the release of the LG12 enzyme;

FIG. 8 is a graph illustrating mg of LG12 released from PSA 7-4602/PVA/colloidal silver patches;

FIG. 9 is an illustration representing LGI2 enzyme release from a PSA/PVA I colloidal silver formulation on a skim milk plate in 24 hours;

FIG. 10 is an illustration representing LG12 enzyme release from a PSA/PVA I DC 5700 formulation on a skim milk plate in 24 hours;

FIG. 11 is a graph illustrating mg of LGI2 released from PSA 7-4602+PVA patch formulation (10-thy incubation at 42° C.);

FIG. 12 is a graph illustrating mg of LG12 released from PSA 7-4602+PVA patch formulation (20-day incubation at 42° C.); and

FIG. 13 is a graph illustrating % Enzyme Released from a PVA+PSA 7-4602 patch formulated with varying levels of DC 3563;

FIG. 14A is a cross-sectional side view of a specific embodiment of the dressing particularly suitable for management of wound exudates and wound eschar debridement, wherein an occlusive layer is disposed between the controlled release layer and the absorbent layer, and an adhesive porous silicone mesh layer is disposed between the absorbent and the occlusive membrane and serves both the adhere the dressing to the skin and the absorbent layer to the dressing.

FIG. 14B is a cross-sectional side view of another specific embodiment of the multi-layer dressing particularly suitable for management of wound exudates and wound eschar debridement, further comprising a backing layer as the outermost layer.

### DETAILED DESCRIPTION OF THE INVENTION

In accordance with the present invention, a controlled-release composition may be used in a variety of topical dressings that may be applied to skin, wounded tissue, and diseased tissue. The topical dressings are multi-layered and allow the active agents to be released and applied to the underlying skin, wounded tissue, and diseased tissue. Additionally, the composition may be used to form ointments, and the ointments allow the active agents to be released and applied to the underlying skin, wounded, or diseased tissue.

A topical dressing shall be understood as referring to any of the various types of coverings that are suitable for application directly to the substrate, e.g. skin, wounded tissue, or diseased tissue for absorption of secretions, protection of the tissue from trauma, administration of an active agent to the tissue, protection of the tissue from the environment, to stop bleeding, to maintain or provide a moist environment, and combinations thereof. For example, the topical dressing may be in the form of films, patches, bandages, gels, ointments, and other semi-solid compositions that, when applied to the skin, dry to form films or coatings on the skin. The topical dressing is multi-layered in the sense that it includes a layer of the controlled-release composition and at least one additional layer, such as an adhesive layer. However, without a multi-layered dressing, the controlled-release composition by itself can form the film on the substrate and functional as a suitable topical application or ‘dressing’.

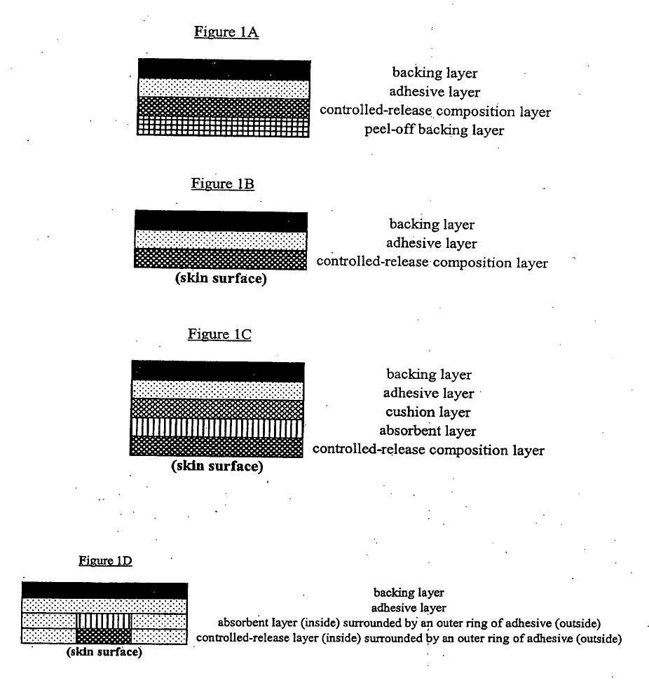

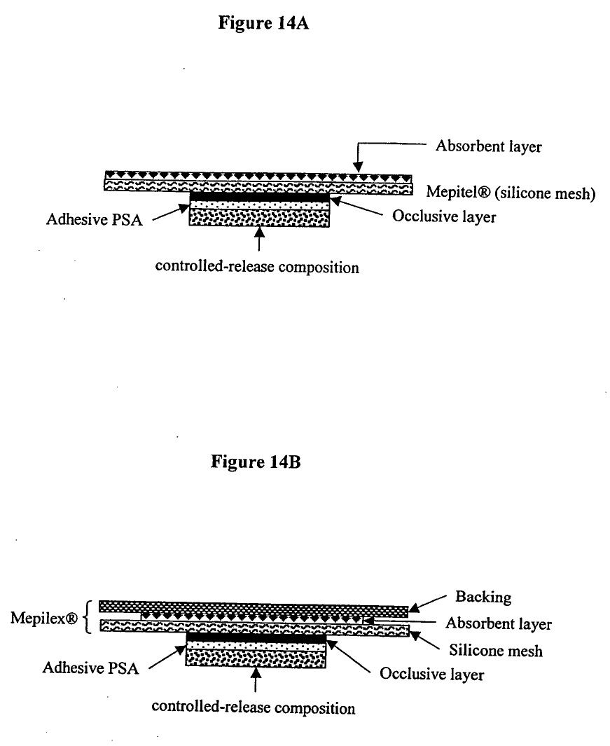

Various forms of the multi-layer dressing are disclosed in FIGS. 1A-1D, and in FIGS. 14A and 14B. The dressing may include as many as about six layers. The various layers are labeled in FIGS. 1A-1D and 14A-14B. With respect to FIG. 1, the adhesive layer (light dots) enhances binding to provide better attachment of the controlled-release composition layer (dark cross-hatch). The controlled-release composition layer may be covered by an absorbent layer (vertical lines) to adsorb exudates and this layer could be overlaid by a cushion or cushioning layer (light cross-hatch). A backing layer (solid black) is occlusive to liquid water. Also, referring specifically to FIG. 1A, before application or adherence to the substrate, i.e., the skin surface, the dressing may also include a peel-off backing layer which is removable. Further, as shown in FIG. 1D, various layers, such as the adhesive layer, could be formed as a ring surrounding other layers.

FIGS. 14A and B illustrate multi layer dressing embodiments particularly suited to management of wound exudates and debridement of wound eschar. In some embodiments, it is desirable to place an occlusive layer (solid black) between the controlled release composition and the absorbent layer. This arrangement directs the controlled release composition toward the wound. Further, an occlusive layer between the controlled-release composition and circulating air and/or an absorbent layer provides a moist wound environment which enhances non-invasive debridement of wound eschar.

The absorbent layer, which is intended to absorb wound exudate, must be accessible to absorb wound exudate from the substrate. However, if wound exudate is absorbed into the absorbent layer, which then remains in contact with the skin, irritation of the healthy skin surrounding the wound but in contact with the dressing, may occur. Hence, in specific embodiments, the absorbent layer may be attached to an adhesive porous layer that serves both to adhere the dressing to the substrate, and to provide a barrier between the exudate absorbed into the absorbent layer, and the substrate, particularly where the substrate comprises tissue susceptible to irritation by the exudate, such as skin. In specific embodiments the adhesive porous layer comprises an adhesive silicone mesh. As depicted for illustrative purposes in FIGS. 14A and 14B, the controlled release layer, the adhesive layer, and the occlusive layer are all substantially co-extensive in area, the area equal to C. In further specific embodiments, the porous adhesive layer and the absorbent layer are substantially co-extensive in area, the area equal to A, and in very specific embodiments, the area A is greater than the area C and extends beyond C to form a border area, B, further wherein the porous adhesive layer contacts and adheres the dressing to the substrate.

In some embodiments, the controlled release layer, the adhesive layer, and the occlusive layer combine to form a patch. The patch is adhered to the silicone mesh/absorbent layer combination, which extends out from the perimeter of the patch forming a border around it. The adhesive silicone mesh adheres to the substrate and surrounds the wound. In this way, wound exudates pass through the porous silicone mesh to the absorbent layer. This arrangement prevents undesirable prolonged contact of the wound exudates with the skin surrounding a wound, and reduces irritation that typically occurs in instances of such prolonged contact. In addition, this arrangement affords the desirable combination of keeping the controlled release composition at the wound site, while permitting the wound exudate to move into the absorbent layer and away from healthy skin.

FIG. 14B illustrates this arrangement with an additional backing layer. In a very specific embodiment, Mepitel®silicone mesh, is employed and it is combined with an absorbent layer as illustrated in FIG. 14A. In another very specific embodiment, Mepilex®border silicone mesh is employed as a combination silicone mesh/absorbent/backing layer, as illustrated in FIG. 14B. Method embodiments provide wound exudate management and debridement of wound eschar. The methods generally comprise the application of inventive dressing embodiments comprising an occlusive layer disposed between the controlled release layer and the absorbent layer, and, in further specific embodiments, comprises a porous adhesive layer disposed between the substrate and the absorbent layer.

Ointment shall be understood as referring to any suitable semi-solid preparation for external application, such as to skin, wounded tissue, and diseased tissue.

The present invention includes a controlled-release composition, essentially an emulsion that has been specifically processed and an active agent, for topical application to a substrate. The present invention also includes a method of delivering the active agent to the substrate. As eluded to above, the controlled-release composition, which is hereinafter simply referred to as the composition, includes the emulsion and the active agent. The active agent is incorporated into the emulsion.

Emulsion shall be understood as referring to a temporary or permanent dispersion of one liquid phase within a second liquid phase. Generally one of the liquids is water or an aqueous solution, and the other is oil or other water-immiscible liquid. Consequently, the continuous or external phase in a water-in-oil emulsion (W/O) is oil or other water-immiscible liquid. The continuous phase in an oil-in-water (O/W) emulsion is water or an aqueous solution. For the descriptive purposes of the present invention, in an O/W emulsion, the term external phase is frequently used interchangeably with hydrophobic phase, and the term internal phase is frequently used interchangeably with hydrophilic phase. Advantageously, the active agent and, if present, the surfactant can be added to the emulsion during various emulsification steps that are undertaken to provide the emulsion or after the emulsion has been provided in a post-add situation without effecting the release profile of the active agent or the overall stability of the emulsion.

Controlled-release shall be understood to means that release kinetics are engineered into the system such that the active agent is released in a manner controlled by the system itself or its surroundings. The agent is not all released within a short period of time, e.g. less than about two hours, but rather is slowly released from a dressing in the presence of a trigger such as moisture over time, e.g. about 4-6 hours, about 4-12 hours, about 4-18 hours, about 4-24 hours, about 4-36 hours, about 4-48 hours. A controlled-release is equivalent to a sustained-release.

Emulsion shall be understood as referring to a temporary or permanent dispersion of one liquid phase within a second liquid phase and encompasses W/O and O/W emulsions. Generally one of the liquids is water or an aqueous solution, and the other is an oil or other water-immiscible liquid. The first liquid is generally referred to as the continuous or external phase. Emulsions can be further classified as either simple emulsions, wherein the dispersed liquid or internal phase is a simple homogeneous liquid, or a more complex emulsion, wherein the dispersed liquid phase is a heterogeneous combination of liquid or solid phases, such as a double emulsion or a multiple-emulsion.

The emulsion is formed by mixing the internal and external phases in any suitable manner to form the preparations of the present invention such as high-shear processing. A preferred method is the mechanical inversion of a water-in-oil (W/O) emulsion as described additionally below, and the formed O/W emulsion may or may not contain lipophilic solvents. Mechanical inversion is also referred to in the art as mechanical inversion emulsification. The W/O emulsion, which is the basis for the O/W emulsion prior to mechanical inversion, includes a silicone component and a surfactant, preferably in a homogenous oil phase, and also includes water. The W/O emulsion is an embodiment that may be used in the dressings of the present invention. The dressing resulting from this emulsion is stable overtime.

The silicone component of the emulsion, may be a hydrophobic or hydrophilic liquid, semi-solid (e.g. wax, gum), or solid. Regardless of whether the silicone component is itself hydrophobic or hydrophilic, the silicone component is contained within the hydrophobic phase of the emulsion. However, it is also possible that the silicone component be present within the hydrophilic phase of the emulsion. Furthermore, it is also possible that hydrophobic silicone components include some hydrophilic substituents and that hydrophilic silicone components include some hydrophobic substituents.

Preferably, the silicone component is a pressure sensitive adhesive (PSA) that is the reaction product of a hydroxy endblocked polydimethylsiloxane polymer and a hydroxy functional silicate resin. Preferably, the hydroxy functional silicate resin is a trimethylsiloxy and hydroxy endblocked silicate resin. The polymer and resin react in a condensation reaction to form the PSA. Although the PSA is most preferred, other forms of the silicone component include a silicone gum, a silicone rubber, a silicone elastomer, a silicone resin, high molecular weight silicones, or mixtures thereof these components. These other forms of the silicone component are possible because they form a film. Along with the active agent, the PSA functions as a bio-adhesive. The advantage of using the PSA as the silicone component is the substantivity that the PSA provides. This substantivity is particularly advantageous in human and veterinary applications that require significant substantivity for the active agent to provide sustained therapeutic effects.

The silicone components that are emulsified according to the mechanical inversion process, specifically the PSA, the silicone gum, the silicone rubber, the silicone elastomer, the silicone resin, and the high molecular weight silicones in the absence of a lipophilic solvent have viscosities up to 5,000,000,000 (5 billion) centipose (cP), preferably of at least 200,000,000 (200 million) centipose (cP) to 2,000,000,000 (2 billion) centipose (cP), and most preferably of at least 1,000,000,000 (1 billion) centipose (cP).

For purposes of this invention, the terms silicone rubber and silicone elastomer are synonymous, at least to the extent that both silicone components are capable of elongation and recovery. In contrast, silicone gums are capable of being stretched, but they do not generally snap back. Silicone gums are the high molecular weight, generally linear, polydiorganosiloxanes that can be converted from their highly viscous plastic state into a predominately elastic state by crosslinking. Silicone gums are often used as one of the main components in the preparation of silicone rubbers and silicone elastomers.

Silicone emulsions are aqueous emulsions of silicone elastomer particles. Removal of water from these emulsions results in either a silicone elastomeric film or particles of silicone elastomer. These emulsions can be prepared by emulsifying reactive silicone polymers and other ingredients such as catalysts or crosslinking compounds in water followed by a suitable vulcanizing (cure or crosslinking) step. Depending upon the emulsification conditions used, these elastomer emulsions can also be made with mean particle sizes that range from approximately 0.1 Oum to 5 Oum. Silicone elastomer emulsions can be considered to include compositions of the type described in U.S. Pat. No. 6,497,894 (issued Dec. 24, 2002) and U.S. Pat. No. 5,321,075 (issued Jun. 14, 1994) and U.S. Pat. No. 4,248,751 (issued Feb. 3, 1981), the disclosures of which are hereby incorporated by reference in theft entirety.

For purposes of this invention therefore, silicone gum can be considered to include compositions of the type described in U.S. Pat. No. 3,692,737 (issued Sep. 19, 1972), U.S. Pat. No. 4,152,416 (issued May 1, 1979), U.S. Pat. No. 4,885,129 (issued Aug. 8, 1989), and U.S. Pat. No. 5,057,240 (issued Oct. 15, 1991), the disclosures of which are hereby incorporated by reference in their entirety.

Silicone rubbers and silicone elastomers can be considered to include compositions of the type described in U.S. Pat. No. 4,882,377 (issued Nov. 21, 1989), U.S. Pat. No. 5,654,362 (issued Aug. 5, 1997), U.S. Pat. No. 5,994,459 (issued Nov. 30, 1999), and U.S. Pat. No. 6,015,858 (issued Jan. 18, 2004), the disclosures of which are hereby incorporated by reference in their entirety.

Silicone resins can be considered to include compositions of the type described in U.S. Pat. No. 2,676,182 (issued Apr. 20, 1954), U.S. Pat. No. 4,310,678 (issued Jan. 12, 1982), U.S. Pat. No. 4,423,095 (issued Dec. 27, 1983), and U.S. Pat. No. 5,356,585 (issued Oct. 18, 1994), the disclosures of which are hereby incorporated by reference in theft entirety.

The silicone resins of the subject invention may also be considered to include MQ resins. The acronym MQ as it relates to silicone resins is derived from the symbols M, D, T, and Q each of which represent a functionality of different types of structural units which may be present in silicone resins containing siloxane units joined by ≡Si—O—Si≡ bonds. The monofunctional (M) unit represents (CH₃)₃SiO_{1/2} and the difunctional (D) unit represents (CH3)₂SiO_{2/2}. The trifunctional (T) unit represents CH₃SiO_{3/2} and results in the formation of branched linear siloxanes. The tetrafunctional (O) unit represents SiO_{4/2}, which results in the formation of crosslinked and resinous silicone compositions. Hence, MQ is used when the siloxane contains all monofunctional M and tetrafunctional Q units, or at least a high percentage of M and Q units such as to render the silicone resinous.

Silicone resins useful herein are non-linear siloxane resins having a glass transition temperature (Tg) above 0° C. Glass transition temperature is the temperature at which an amorphous material such as a higher silicone polymer changes from a brittle vitreous state to a plastic state. Thin silicone resin generally has the formula R′ₐSiO_{(4-a)/2} wherein R′ is a monovalent hydrocarbon group with 1-6 carbon atoms or a functionally substituted hydrocarbon group with 1-6 carbon atoms, and a has an average value of 1-1.8. The silicone resin will preferably include monofunctional (M) units R″₃SiO_{1/2} and tetrafunctional (Q) units SiO412, in which R′ is the monovalent hydrocarbon group having 1-6 carbon atoms, most preferably the methyl group. Typically, the number ratio of M groups to Q groups will be in the range of 0.5:1 to 1.2:1, so as to provide an equivalent wherein a in the formula R′ₐSiO_{(4.a)/2} has an average value of 1.0-1.63. Preferably, the number ratio is 0.6:1 to 0.9:1. Most preferred are silicone MQ resins in which the number of Q units per molecule is higher than 1, preferably higher than 5.

The silicone resin may also contain 1-5 percent by weight of silicon-bonded hydroxyl radicals such as a dimethylhydroxysiloxy unit (HO)(CH₃)₂SiO_{1/2}. If desired, the silicone resin may contain minor amounts of difunctional (D) units and/or trifunctional (T) units. The silicone resin may include (i) silicone resins of the type MₓQ_{y} where x and y have values such that the silicone resin contains at least more than 5 Q units per molecule; (ii) silicone resins of the type MₓT_{y} where x and y have values such that the silicone resin contains at least more than 5 T units per molecule; and (iii) silicone resins of the type MₓD_{y}TₚQ_{q} where x, y, p, and q have values such that the sum of Q and T units is at least more than 5 units per molecule, and the number of D units varies from 0-100.

As set forth above, the emulsion may include a surfactant. Surfactant shall be understood as referring to a surface-active agent added to a suspending medium to promote uniform and maximum separation of immiscible liquids or liquids and extremely fine solid particles, often of colloidal size. As is understood by those skilled in the art, surfactants are amphiphilic molecules that have polar head groups and nonpolar chains. As such, the surfactants accumulate at the interfaces of the hydrophilic and hydrophobic phases and the polar heads orient toward the hydrophilic phase and the nonpolar chains orient toward the hydrophobic phase. Surfactants promote wetting, efficient distribution of immiscible liquids, droplets, or fine solid particles in a liquid dispersing medium and stabilization against particle aggregation. The surfactant is generally added in the dispersing medium in amount sufficient to provide complete surface coverage of the particle surface. The surfactant may be an anionic surfactant, cationic surfactant, nonionic surfactant, amphoteric surfactant, or a mixture of these surfactants.

Representative examples of suitable anionic surfactants include alkali metal salts of higher fatty acids, alicylaryl sulphonates such as sodium dodecyl benzene sulphonate, long chain fatty alcohol sulphates, olefin sulphates and olefin sulphonates, sulphated monoglycerides, sulphated esters, sulphonated ethoxylated alcohols, sulphosuccinates, alkane sulphonates, phosphate esters, ailcyl isethionates, alkyl taurates, and alkyl sarcosinates. One example of a preferred anionic surfactant is sold commercially under the name Bjo-Soft N-300. It is a triethanolamine salt of dodecylbenzene sulfonic acid marketed by the Stephan Company, Northfield, Ill.

Representative examples of suitable cationic surfactants include alkylamine salts, quaternary ammonium salts, sulphonium salts, and phosphonium salts.

One example of a preferred cationic surfactant is cetyltrimethylammonium chloride sold commercially under the name Anunonxy CETAC 30 marketed by the Stephan Company. Mother example is a quaternary ammonium-functional silane sold commercially under the name DC 5700 marketed by the Aegis Company.

Representative examples of suitable nonionic surfactants include condensates of ethylene oxide with long chain fatty alcohol or fatty acids such as a C₁₂₋₁₆ alcohol, condensates of ethylene oxide with an amine or an amide, condensation products of ethylene and propylene oxide, esters of glycerol, sucrose, sorbitol, fatty acid alkylol amides, sucrose esters, fluoro-surfactants, and fatty amine oxides. Representative examples of suitable amphoteric surfactants include imidazoline compounds, alkylaminoacid salts, and betaines.

Representative examples of suitable commercially available nonionic surfactants include polyvinyl alcohol (PVA or PVOH) (such as, for example, Mowiol® 3-83 and 30-92 available from Clamant Corporation, Charlotte, N.C.) and polyoxyethylene fatty alcohols sold under the tradename BRIJ by Uniqema UCI Surfactants), Wilmington, Del. Some examples are BRIJ 35 Liquid, an ethoxylated alcohol known as polyoxyethylene (23) lauryl ether, and BRIJ 30, another ethoxylated alcohol known as polyoxyethylene (4) lauryl ether. Some additional nonionic surfactants include ethoxylated alcohols sold under the trademark TERGITOL® by The Dow Chemical Company, Midland, Mich. Some example are TERGITOL® TMN-6, an ethoxylated alcohol known as ethoxylated trimethylnonanol; and various of the ethoxylated alcohols, i.e., C₁₂-C₁₄ secondary alcohol ethoxylates, sold under the trademarks TERGITOL® 15-S-5, TERGITOL® 15-S-12, TERGITOL® 15-S-15, and TERGITOL® 15-S-40. Surfactants containing silicon atoms such as silicone polyethers can also be used.

Upon the providing of the emulsion, which includes the silicone component, and optionally the surfactant and the water, the active agent is incorporated, or dispersed, into the emulsion for delivery of the active agent to the substrate upon application of the emulsion to the substrate. Although the active agent may be in powder form or crystalline form, it is typically in liquid or viscous form. The active agent can be post-added into the emulsion whether or not it is combined with a hydrophilic carrier and/or hydrophilic component. Alternatively, the active agent can be incorporated during the steps to provide the emulsion.

Hydrophilic carrier shall be understood as referring to at least one component of a phase of the preparations of the present invention that acts as the solvent for the active agents. The hydrophilic carrier aids in the release of the active agent from the silicone matrices used in embodiments of the present invention.

Hydrophilic component shall be understood as referring to at least one component added to the mixture of the hydrophilic carrier and active agent in embodiments of the present invention. The hydrophilic component may aid in the release of the active agent from the silicone matrices used in embodiments of the present invention.

Active Agent shall be understood as referring to proteins, and in particular to enzymes.

Protein shall be understood as referring to natural, synthetic, and engineered enzymes such as oxidoreductases, transferases, isomerases, ligases, hydrolases; antibodies; polypeptides; peptides; hormones; cytokines; growth factors; and other biological modulators.

The active agents of the present invention are generally proteins, such as enzymes, that are incorporated into the hydrophilic carrier. The active agents may be hydrophilic. Enzymes suitable for incorporation in the dressing may be any enzyme or enzymes. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases and mixtures thereof.

Lipase enzymes which may be considered to be suitable for inclusion in the preparations of the present invention include those produced by microorganisms of the Pseudomonas group, such as Pseudomonas stutzeri ATCC 19.154, as disclosed in British Patent 1,372,034; Pseudomonas tnendocina, as described in U.S. Pat. No. 5,389,536, and Pseudomonas pseudoalcaligenes, as disclosed in U.S. Pat. No. 5,153,135. Lipases further include those that show a positive immunological cross-reaction with the antibody of the lipase, produced by the microorganism Pseudomonas fluorescens IAM 1057. This lipase is available from Amano Pharmaceutical Co. Ltd., Nagoya, Japan, under the trade name Lipase P “Amano”. Lipases include M1 Lipase® and Lipomax® (Gist-Brocades NV, Delft, Netherlands) and Lipolase® (Novozymes A/S, Bagsvaerd, Denmark). The lipases are normally incorporated in the silicone matrix at levels from about 0.0001% to about 2% of active enzyme by weight of the silicone matrix, or from about 0.001 mg/g to about 20 mg/g.

Proteases are carbonyl hydrolases which generally act to cleave peptide bonds of proteins or peptides. As used herein, “protease” means a naturally-occurring protease or a recombinant protease. Naturally-occurring proteases include .alpha.-aminoacylpeptide hydrolase, peptidylamino acid hydrolase, acylamino hydrolase, serine carboxypeptidase, metallocarboxypeptidase, thiol proteinase, carboxylproteinase and metalloproteinase. Serine, metallo, thiol and acid proteases are included, as well as endo and exo-proteases.

The protease can be of animal, plant, or microorganism origin. For example, the protease may be a serine proteolytic enzyme of bacterial origin. Purified or nonpurified forms of enzyme may be used. Protease enzymes produced by chemically or genetically modified mutants are included by definition, as are close structural enzyme variants. Particularly preferred by way of protease enzyme is bacterial serine proteolytic enzyme obtained from Bacillus, particularly subtilases, for example Bacillus subtilis, Bacillus lentus, Bacillus amyloliquefaciens, and/or Bacillus licheniformis. Suitable commercial proteolytic enzymes which may be considered for inclusion in the present invention compositions include Alcalase®, Esperase®, Durazym®, Everlase®, Kannase®, Relase®, Savinase®, Maxatase®, Maxacal®, and Maxapem® 15 (protein engineered Maxacal); Purafect®, Properase® (protein engineered Purafect) and subtilisin BPN and BPN′.

Protease enzymes also encompass protease variants having an amino acid sequence not found in nature, which is derived from a precursor protease by substituting a different amino acid sequence not found in nature, which is derived from a precursor protease by substituting a different amino acid for the amino acid residue at a position in said protease equivalent to positions equivalent to those selected from the group consisting of +76, +87, +99, +101, +103, +104, +107, +123, +27, +105, +109, +126, +128, +135, +156, +166, +195, +197, +204, +206, +210, +216, +217, +218, +222, +260, +265, and/or +274 according to the numbering of Bacillus amyloliquefaciens subtilisin, as described in U.S. Patent Nos. RE 34,606; U.S. Pat. No. 5,700,676; U.S. Pat. No. 5,972,682 and/or U.S. Pat. No. 6,482,628, which are incorporated herein by reference in their entirety.

Exemplary protease variants include a subtilisin variant derived from Bacillus lentus, as described in U.S. Patent No. RE 34,606, hereinafter referred to as Protease A. Another suitable protease is a Y217L variant derived from Bacillus amyloliquesfaciens, as described in U.S. Pat. No. 5,700,676, hereinafter referred to as Protease B. Also suitable are what are called herein Protease C, which is a modified bacterial serine proteolytic enzyme described in U.S. Pat. No. 6,482,628; and Protease D, which is a modified bacterial serine proteolytic enzyme described in U.S. Pat. No. 5,972,682. Also suitable is LG12 a B. subtilis as described in U.S. Pat. No. 5,677,163, which is incorporated by reference herein.

Other proteases useful in the practice of this invention can be selected from the group consisting of Savinase®, Esperase®, Maxacal®, Purafect®, BPN′, Protease A, Protease B, Protease C, Protease D, LG12 and mixtures thereof. Protease enzymes are generally present in the preparations of the present invention at levels from about 0.0001% to about 2% of active enzyme by weight of the silicone matrix, or from about 0.00 1 mg/g to about 20 mg/g.

It will be understood by those having skill in the art that the present invention is not limited to the enzymes listed above. It shall be further understood by those having skill in the art that one or more active agents including non-proteinaceous active agents such as anti-infection and biocide agents can be utilized in the topical preparations of the present invention.

The active agents, and any non-proteinaceous agents, may perform a variety of functions. For example, the matrix can release proteases and other enzymatic debriding agents topically for removal of necrotic tissues and general wound cleansing, clotting formation and clot removal enzymes, agents which generate peroxide, peracid, activated oxygen species, and anti-adhesion catalytic antagonists for self-sterilization, anti-infection, and acceleration of healing, and agents for skin treatment and the like.

Additionally, hydrophilic and/or amphiphilic excipients can be employed to stabilize or compatibilize the active agents, as well as assist in theft release from the silicone matrix. Excipients can be liquid, semi-solid (e.g. wax, gum), or solid. Silicone excipients for use with the present invention can include silicone polyethers, silicone fluids, dimethicones, dimethicone copolyols, dimethiconols, silicone alkyl waxes, silicone polyamides and the like. Other possible excipients include, but are not limited to, silver, (poly)saccharide derivatives, acrylate derivatives, PVA derivatives, glycol, glycerol, glyceride derivatives, propylene glycol (PPG), polyethylene glycol, poloxamer, glycerin, alcohol, cellulosic derivatives, polyacrylic acids, alginate derivatives, chitosan derivatives, gelatin, pectin and polyhydric alcohol.

Also, various cosmetic, personal care, and cosmeceutical components may be included aside from the excipient or excipients. Examples of suitable cosmetic, personal care, and cosmeceutical components include, but are not limited to, alcohols, fatty alcohols and polyols, aldehydes, alkanolamines, alkoxylated alcohols (e.g. polyethylene glycol derivatives of alcohols and fatty alcohols), alkoxylated amides, alkoxylated amines, alkoxylated carboxylic acids, amides including salts (e.g. ceramides), amines, amino acids including salts and alkyl substituted derivatives, esters, alkyl substituted and acyl derivatives, polyacrylic acids, acrylamide copolymers, adipic acid copolymers, alcohols, aminosilicones, biological polymers and derivatives, butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (clay, TiO2, ZnO), ketones (e.g. camphor), isethionates, lanolin and derivatives, organic salts, phenols including salts (e.g. parabens), phosphorus compounds (e.g. phosphate derivatives), polyacrylates and acrylate copolymers, protein and enzymes derivatives (e.g. collagen), synthetic polymers including salts, siloxanes and silanes, sorbitan derivatives, sterols, sulfonic acids and derivatives and waxes.

The method of the subject invention includes more specific steps in order to provide the emulsion. In accordance with a preferred embodiment, a preparation is provided comprising an internal or non-miscible dispersed phase within an external or continuous phase. The hydrophobic phase generally comprises a silicone matrix, and the hydrophilic phase generally comprises a hydrophilic carrier containing at least one active agent. Additionally, the hydrophilic phase may further comprise any suitable hydrophilic component.

The hydrophilic phase may comprise any suitable hydrophilic carrier containing at least one active agent. In an embodiment according to the invention, the hydrophilic carrier is a liquid at relevant temperatures, and solid materials (for example sorbitol, manitol, lactose, sodium chloride and citric acid) dissolved in suitable solvent also may be used. For example, the active agent may be contained in a solution of propylene glycol (PPG), polyethylene glycol, poloxamer, glycerin, alcohol, polyhydric alcohol, water, or other suitable hydrophilic carrier.

The hydrophilic phase may further comprise a water soluble and hydrophilic component. The hydrophilic component generally does not serve as a solvent for the active agent. The hydrophilic component may enhance the release rate of the active agent from the silicone matrix and can include polyvinyl alcohol (PVA or PVOH) (such as, for example, Mowiol® 3-83 and 30-92 available from Clariant Corporation, Charlotte, N.C.). The hydrophilic phase solution can include up to about 50 or more wt. % PVA solution in water. However, it is understood that both higher molecular weight and increased concentration of PVA result in a higher viscosity of the final composition. In an embodiment according to the invention, the hydrophilie component can also be a water-thickening agent diluted in water such as cellulosic derivatives (such as carboxymethylcellulose, methylcellulose, sodium carboxymethyl cellulose, hydroxypropylcellulose, hydroxypropylmethylcellulose), polyacrylic acids, alginate derivatives, chitosan derivatives, gelatin, pectin, polyethylene glycol, propylene glycol, glycerol and other suitable hydrophilic molecules and macromolecules in which the active agent may or may not be soluble. Such molecules include hydrophilic macromolecules.

The emulsion is formed by mixing the internal and external phases in any suitable manner to form the preparations of the present invention such as high-shear processing. Preferably, the W/O emulsion is mechanically-inverted into an O/W emulsion. This mechanical inversion is preferably accomplished by applying a high shear to the W/O emulsion. Suitable high-shear equipment include a high-intensity mixer, homogenizer, colloidal mill, Sonolator, Microfluidizer, ultrasonic processor, change can mixer, and a generic dental mixer such as Hauschild SpeedMixer™ supplied by Flacktek. The preferred mixing device (i.e. dental mixer) consists of a mixer enclosed in a housing and having a motorized arm that rotates about a first axis of rotation, and a basket arranged to rotate about a second axis of rotation, in the opposite direction while the arm is rotating. During operation the basket rotates around an axis in one direction while it is simultaneously rotating (oppositely) in a planaterary motion around another axis. A container holding the substances to be mixed is placed in the basket and the mixer is energized for a period of time, which is controlled by an electronic timer. As the mixing is very efficient, the normal mixing periods are typically on the order of 20 seconds. This mixer is sold commercially by the name of SpeedMixer™ by Flacktek, Inc., Landrum, S.C. A description of this mixer may be found in U.S. Pat. No. 6,755,565 (issued Jun. 29, 2004).

Inversions generally occur when the continuous phase of a dispersion becomes the dispersed phase, or vice versa. Phase inversions in liquid/liquid dispersions are categorized as either catastrophic inversions or transitional inversions. Catastrophic inversions are caused by simply changing the phase ratio until there is a high enough ratio of the dispersed phase that it becomes the continuous phase. Transitional inversions occur when the affinity of the surfactant for the two phases is altered in order to cause the inversion.

After inversion, the O/W emulsion may then be diluted with additional water. If added, the additional water is typically added after a desired particle size for the silicone component has been reached. The droplet size of the internal phase may vary. For example, the droplet size in the preferred embodiment may be from about 0.01 μm up to about 1000 μm, while the most preferred embodiment is from about 0.1 μm up to about 0.5 μm.

The solids content may be selectively varied to achieve a target viscosity for ideal application of the emulsion to the substrate or to effect the rate of delivery of the active agent to the substrate.

As alluded to above, the active agent can be incorporated during the steps that are undertaken to provide the emulsion. More specifically, the active agent may be incorporated into the emulsion by incorporating the active agent along with the step of adding the water to the emulsion containing the continuous phase and the dispersed phase. The emulsion can contain other additives including, but not limited to, biocides (e.g. DC 5700), silver, thickeners, freeze-thaw stabilizers and electrically conductive additives, such as an ionic species, to make a conductive emulsion that can be used as electrodes in electrophoretic applications.

One embodiment provides a method of delivering the active agent to the substrate further including the step of applying the emulsion to the substrate to deliver the active agent to the substrate. Upon application of the emulsion, which contains the active agent, and upon exposure of the substrate to air, the solvent leaves the emulsion and a film is formed on the substrate. The film contains the active agent.

In sum, one delivery method provides at least one layer of the dressing, then provides the emulsion, oil-in-water or water-in-oil, then incorporates the active agent comprising the protein into the emulsion to establish the controlled-release composition, and the controlled-release composition is applied to the layer to form a controlled-release layer of the dressing. Upon processing, the controlled-release composition may be dried such that the controlled-release layer is free of water or can contain up to 15% of water. Free of water can be understood to include water or free of all water, with the exception of any water inherently bound to the enzyme.

In embodiments where the substrate is skin, the emulsion is applied to the skin to deliver the active agent to the skin. The emulsion may be applied, i.e., rubbed or coated, directly onto the skin. Alternatively, the emulsion may be incorporated into a bandage or patch dressing prior to application to the substrate, i.e., to the skin.

The controlled-release composition according to this invention is capable of delivering performance properties such as adhesion, controlled tack, controlled lubrication, shear reduction, cushioning, water resistance, barrier properties, maintenance or provision of a moist wound environment, and scar-reduction. This controlled-release composition has substantivity to the skin and other substrates. In addition, an adhesive substance can be applied to a transdermal patch to improve adhesion if desired. The significant substantivity of the composition is particularly advantageous when delivery of the active agent is required over an extended period of time. Simply stated, the controlled-release composition is topically applied to the substrate where the film remains over the extended period of time. When the substrate is skin, the substantivity is important due to the presence of certain body oils and especially upon application to hairy skin. The composition also has substantivity to wet substrates such as wounds.

The topical dressing may be a liquid, semi-solid, or solid.

Since the topical dressing is understood as referring to any of the various types of coverings, a liquid or semi-solid dressing may be in the form of an ointment, gel, foam, and a low viscosity fluid. Such dressings could be packaged and delivered from a tube, syringe, stick, pump, spray, or a wipe and combinations thereof. The liquid form can remain as a liquid, such as an ointment dressing, or solidify during the formation of a film due to evaporation or cross-linking such as a liquid bandage.

A solid dressing has a three dimensional form such as an adhesive strip, bandage, putty, or a single or multi-layer film or membrane (e.g. transdermal patch). As illustrated in FIG. 1, a three-dimensional solid dressing may include an adhesive, controlled-release composition, absorbent, cushion, occlusive, or a backing material and combinations thereof. The solid dressing may utilize an outer adhesive layer that extends beyond the outer margin of the controlled-release composition layer, and/or adhesive material may be positioned on the skin facing controlled-release composition layer, but along the outer margins of this layer. The multi-layer solid dressing may be in the form of continuous or discrete layers, dots, adhesive rim, or pattern coated network layer including open space and combinations thereof. The solid dressing may have any type of shape, thickness, and size. It can be highly flexible or rigid. It can be self-adhering (e.g. a full adhesive surface or adhesive rim) or require a secondary dressing or bandage to remain in place. It can be self-supported, impregnated into a textile (e.g. gauze, Dacron net, knitted fabric), and/or reinforced with an additional backing material. A natural (e.g. collagen, alginate, cellulose) or synthetic (e.g. plastic and elastomeric films) backing material may be a non-woven material or a knitted textile, transparent or opaque, perforated, plain, embossed, or cellular (e.g. foam) and combinations thereof. For example, plastic and elastomeric films include semi-occlusive polyurethanes, polyethylene, and silicone membrane, such as Dow Corning® 7-4107. Occlusive polyurethanes include, by non-limiting example, Medifilm® 437 from Mylan Technologies, Bioflex 130 from Scapa Medical, and Vacuflex® from Omniflex.

The controlled-release composition is composed of preparations comprising silicone matrices and hydrophilic carriers that provide controlled-release of active agents. The continuous or discrete layers within a multi-layer solid dressing may provide multiple functions. For example, a controlled-release composition may also have adhesive, absorbent, cushion, or barrier properties and combinations thereof.

In some embodiments, an adhesive layer serves to adhere the controlled-release layer to other layers. In one embodiment of the multi-layer dressing, the adhesive layer comprises a silicone adhesive, a non-silicone adhesive, or combinations thereof. In a specific embodiment, the non-silicone adhesive is selected from the group consisting of acrylic adhesive, rubber adhesive, and combinations thereof, and in a more specific embodiment, the non-silicone adhesive comprises an acrylic adhesive. Acrylic adhesives comprise acrylic polymers and an aqueous or nonaqueous solvent. In a specific embodiment, the acrylic adhesive comprises acrylic polymers and an aqueous solvent. In more specific embodiments the acrylic adhesive is selected from the group consisting of DC®MG-0560, and DC® MG-0580, and in other very specific embodiments the acrylic adhesive is selected from the group consisting of DC® MG-0610 and DC® MG-0607.

In embodiments of the multi-layer dressing wherein the adhesive layer comprises a non-silicone adhesive, the non-silicone adhesive may comprise a rubber adhesive. Rubber adhesives particularly suitable for employment as the adhesive according to the present invention comprise a styrene-butadiene copolymer. In very specific embodiments the rubber adhesive comprises DC® MG-0156.

The absorbent layer absorbs exudate fluids from wounds. In certain embodiments particularly suited for management of wound exudates, the absorbent layer is separated from the substrate, for example, skin, by a porous adhesive layer. The porous adhesive layer permits wound exudates to penetrate to the absorbent layer and prevents prolonged contact of the exudates with skin, reducing and even eliminating skin irritation. The porous adhesive layer also adheres the absorbent layer to the other layers of multi-layer dressing, while, in some embodiments, further serving to adhere the multi-layer dressing to the skin. In one embodiment of the multi-layer dressing, the porous adhesive layer comprises an adhesive silicone mesh. Particularly exemplary silicone meshes include the proprietary silicone meshes Mepitel®, which is a silicone mesh layer, and Mepilex®border, which is a combination backing/absorbent/silicone mesh layer.

In one embodiment particularly suited to the care of chronic wounds, the multi layer dressing provides both effective management of wound exudate and effective debridement of wound eschar. In this embodiment, an occlusive layer is disposed between the controlled release layer and the absorbent layer, directing the controlled release layer to the wound while maintaining a moist wound environment. Further, a porous adhesive layer is disposed between the substrate and the absorbent layer, so that wound exudate is directed away from the substrate, in particular, healthy skin surrounding a wound, and toward the absorbent layer. Wound exudate is effectively managed according to the present invention when the skin surrounding a wound remains substantially free of irritation and accompanying discoloration while the dressing is in place. Debridement of wound eschar is effective according to the present invention if debridement is substantially completed and wound exudate is effectively managed. Certain embodiments of the invention contemplate effective debridement of a wound after application of a multi-layer dressing according to the present invention, to the wound, for a period of less than about 48 hours. In more specific embodiments, effective debridement occurs after application for a period of less than about 36 hours, and in very specific embodiments, effective debridement occurs after a period of about 24 hours.

Cushioning layers provide padding over wounds, such as diabetic foot ulcers, to prevent re-injury. The backing layer may be occlusive to liquids and provide structural support for the dressing. A solid dressing may be constructed using any type of process to transform and give shape to liquid or plastic materials including coating, casting, injection-molding, and extrusion. The final device may be made by punching it into a multi-layer sheet, molding it directly into the final packaging (e.g. blister), or assembling it from distinct pieces and combinations thereof.

Normal wounds progress through a series of stages where the processes of healing in different stages overlap. Typically, there is a cleansing phase where necrotic and damaged tissues are removed by cellular and enzymatic processes, followed by a granulation phase where growth hormones are produced. There is increased blood vessel formation and the migration of a series of specialized cells such as fibroblasts that begin to create a scaffold of fibrin and subsequently collagen which serves as a support structure for the final phase of epithelialization which results in the closure of the wound. Chronic wounds, as used herein, are those wounds that do not heal and are believed to be stalled in the healing process. Without being bound by theory, a key to effective healing is believed to be removal of the necrotic tissue present in a wound. Debridement, as used herein, is the process of removing the necrotic material. Conventional methods depend on the necrotic tissue being removed by a scalpel. An alternative to surgical debridement is chemical debridement using enzymes, especially, proteolytic enzymes. Most commercially known products require numerous applications, however. Non-invasive, rapid debridement of eschar in chronic wounds such as pressure ulcers, venous leg ulcers, and diabetic fool ulcers represents a particular unmet need.

The present invention provides methods directed to managing wound exudate and wound debridement. One embodiment of the present invention is directed to methods of effectively managing wound exudates and/or of achieving effective debridement of wound eschar. The method comprises: adhering a multilayer dressing to a substrate for an effective amount of time, and the multilayer dressing comprises: (A) a controlled-release layer formed from a controlled-release composition comprising: (i) an oil-in-water or water-in-oil emulsion, and (ii) an active agent incorporated into said emulsion and comprising a protein; and (B) an adhesive layer disposed adjacent said controlled-release layer; (C) an occlusive layer disposed adjacent said adhesive layer and away from said controlled release layer; (E) an absorbent layer disposed opposite the occlusive layer from the controlled release layer; and, (F) optionally, at least one additional layer selected from the group of a backing layer, a cushioning layer, a second absorbent layer, a second adhesive layer, a porous adhesive layer, and combinations thereof. By placing the occlusive layer between the controlled-release layer and the absorbent layer, the controlled release composition is directed toward the wound and the wound environment is kept moist. In another specific embodiment, the dressing comprises a porous silicone adhesive layer which is permeable to wound exudate, directing it toward the absorbent layer and away from the substrate. In applications where the substrate is sensitive to the exudate, skin, for example, this prevents prolonged contact of the exudate with the skin, lessening the frequency of necessary dressing changes and hastening healing of the wound. In specific embodiments, the porous adhesive layer comprises a silicone mesh, and in a very specific embodiment, the silicone mesh comprises Mepitel® silicone mesh.

In order that the invention may be more readily understood, reference is made to the following examples, which are intended to be illustrative of the invention, but are not intended to be limiting in scope.

### EXAMPLES

### Example 1

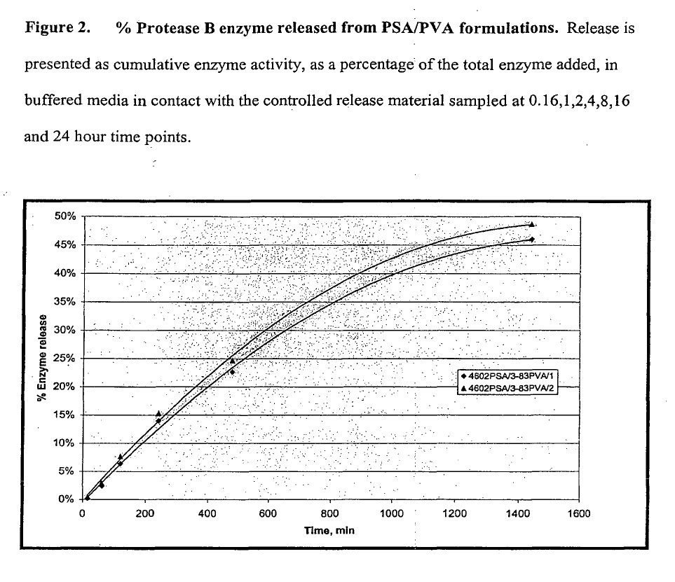

This experiment was conducted to evaluate the sustained release of Protease B enzyme from a silicone matrix. First the hydrophilic phase was prepared by mixing 8.71 g of hydrophilic carrier PVA solution (40% Mowiol 3-83 in water) with 0.767 ml of Protease B enzyme 42 mg/ml stock solution. Then, 20.43 g of the silicone phase was added to this mixture. The silicone matrix in this case was Dow Corning® PSA 7-4602 a pressure sensitive adhesive. After each addition step the sample was mixed two times in a Houschild AM-501 dental mixer. The prepared emulsions were spreaded on DC 7-4107 silicone membrane/Polycarbonate substrate using a draw down bar made by Paul N. Gardner Company, Inc. The drawn film was allowed to thy to a thin film on the substrate over 24 hours in a ventilated hood. From the dried film, patches were cut out and analyzed for enzyme release activity. The samples were tested using Franz Cell Assembly and N-succinyl-L-Ala-L-Ala-L-Pro-L-Phe-p-nitroanilide (suc-AAPF-pNA) assay for proteolytic activity. The Franz Cell body was filled with dissolution buffer (10 mM MES, 10 mM CaCl2, and 0.005% Tween 80 at pH 5.4) and a patch sample was attached on top of the cell. Samples from the cell were collected after 15 minutes, 1 hour, 2 hours, 4 hours, 8 hours and 24 hours. From each collected samples, aliquots were pipetted directly into a cuvette containing assay buffer (100 mM Tris and 0.005% Tween 80 at pH 8.6) and suc-AAPF-pNA substrate. Then, the enzyme activity was measured on a UV/Visible spectrometer, which gave the concentration of enzyme in the dissolution buffer in mg/ml. FIG. 2 illustrates the results of the enzyme release from this matrix with Protease B enzyme. A controlled-release of about 50% Protease B was observed over 24 hours.

### Example 2

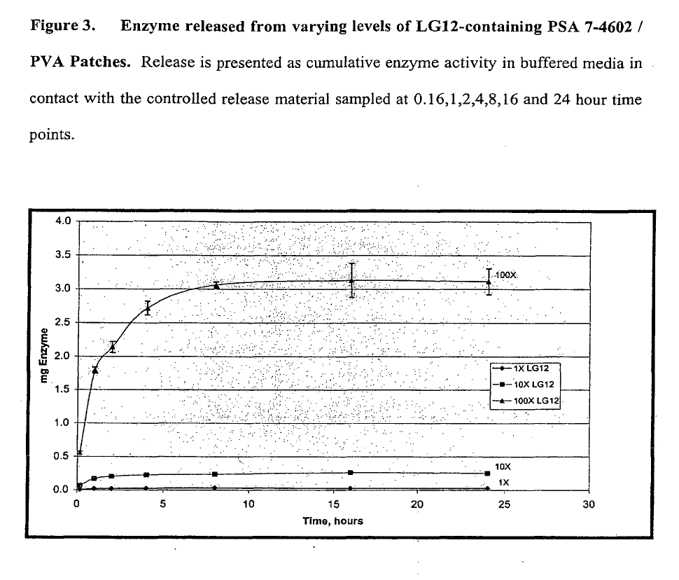

This experiment was conducted to evaluate the sustained release of LG 12 protease from a silicone matrix using 1×, 10× and 100× enzyme loadings. First, the hydrophilic phase was prepared by mixing 13 g of hydrophilic carrier PVA solution (40% Mowiol 3-83 in water) with 3.192 ml LG-12 protease enzyme stock solution. Then, 30 g of silicone phase was added to this mixture. The enzyme stock solution was 0.4098 mg/ml in case of 1×, 4.098 mg/ml in case of 10×, and 40.98 mg/ml in case of 100× enzyme loading. The silicone matrix in this case was Dow Corning® PSA 7-4602 a pressure sensitive adhesive. After each addition step, the sample was mixed two times in a Houschild AM-501 I dental mixer. The prepared emulsions were spreaded on DC 7-4107 silicone membrane/Polycarbonate substrate using a draw down bar made by Paul N. Gardner Company, Inc. The drawn film was allowed to dry to a thin film on the substrate over 24 hours in a ventilated hood. From the dried film, patches were cut out and analyzed for enzyme release activity. The samples were tested using Hanson SR8 Plus Dissolution Tester and N-succinyl-L-Ala-L-Ala-L-Pro-L-Phe-p-nitroanilide (sucAAPF-pNA) assay for proteolytic activity. The Hanson SR8 Pius Dissolution tester was filled with dissolution buffer (10 mM MES, 10 mM CaCl2, and 0.005% Tween 80 at pH 5.4) and a patch sample was placed inside of the vessel. Samples from the cell were collected after 10 minutes, 1 hour, 2 hours, 4 hours, 8 hours, 16 hours and 24 hours. From each collected sample, aliquots were pipetted directly into a cuvette containing assay buffer (100 mM Tris and 0.005% Tween 80 at pH 8.6) and suc-AAPF-pNA substrate. Then, the enzyme activity was measured on a UV/Visible spectrometer, which gave the concentration of enzyme in the dissolution buffer in mg/ml. FIG. 3 illustrates the results of the enzyme release from this matrix. Complete release of LG12 protease was observed over 24 hours. By increasing the enzyme load in the formulation, the enzyme release from the patches can be enhanced proportionally.

### Example 3

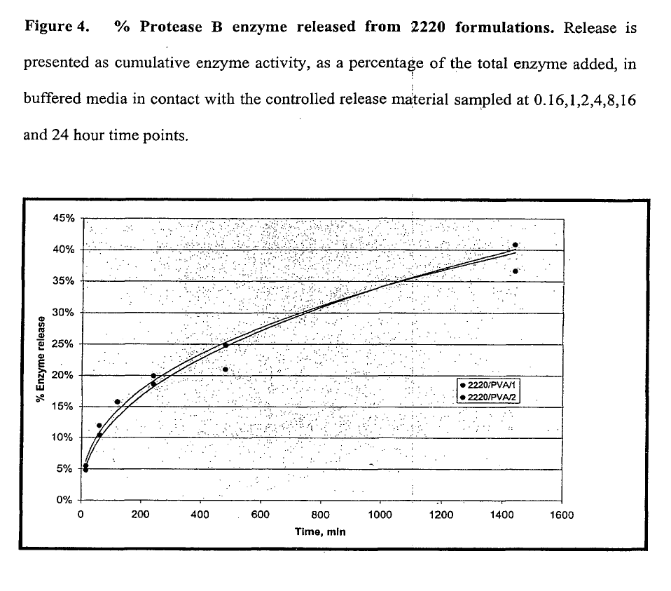

This experiment was conducted to evaluate the sustained release of Protease B enzyme from a silicone matrix. First, the hydrophilic phase was prepared by mixing 17.4 g of hydrophilic carrier PVA solution (10% Mowiol 30-92) with 0.42 g of Protease B enzyme 42 mg/ml stock solution. Then, 10.04 g of silicone phase was added to this mixture. The silicone matrix in this case was Dow Corning® high molecular weight 2220 non-ionic emulsion. After each addition step, the sample was mixed two times in a Houschild AM-501 dental mixer. The prepared emulsions were spread on DC 7-4107 silicone membrane/Polycarbonate substrate using a draw down bar made by Paul N. Gardner Company, Inc. The drawn film was allowed to dry to a thin film on the substrate over 24 hours in a ventilated hood. From the dried film, patches were cut out and analyzed for enzyme release activity. The samples were tested using Franz Cell Assembly and N-succinyl-L-Ala-L-Ala-L-Pro-L-Phe-p-nitroanilide (suc-AAPF-pNA) assay for proteolytic activity. The Franz Cell body was filled with dissolution buffer (10 mM MES, 10 mM CaCI2, and 0.005% Tween 80 at pH 5.4) and a patch sample was attached on top of the cell. Samples from the cell were collected after 15 minutes, 1 hour, 2 hours, 4 hours, 8 hours and 24 hours. From each collected sample, aliquots were pipetted directly into a cuvette containing assay buffer (100 mM Tris and 0.005% Tween 80 at pH 8.6) and suc-AAPF-pNA substrate. Then, the enzyme activity was measured on a UV/Visible spectrometer, which gave the concentration of enzyme in the dissolution buffer in mg/ml. FIG. 4 illustrates the results of the enzyme release from this matrix. A controlled-release of about 40% Protease B was observed over 24 hours.

### Example 4

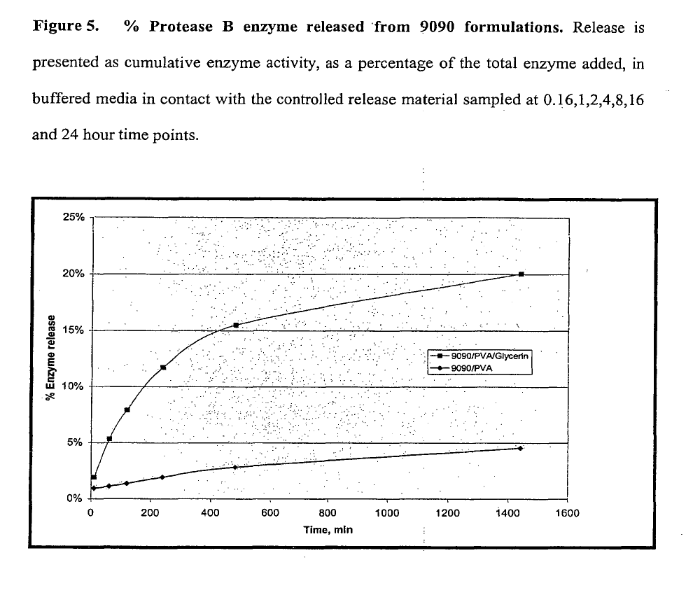

This experiment was conducted to evaluate the sustained release of Protease B enzyme from a crosslinked silicone matrix as well as assess the role of glycerin in the formulation. First, 0.49 g of Dow Corning® 1-3502 Si—H fluid was incorporated into 60 g of Dow Corning® SFD 128 vinyl silicone polymer. Subsequently, 10.08 g of 10% Mowiol (30-92) surfactant was added to the mixture until a high solid emulsion was formed. Then, 0.44 g of Pt catalyst was mixed into the formulation in emulsion form (Dow Corning® 2-1271). This formulation is analogous to a Dow Corning® 9090 Silicone Elastomer Emulsion. Then, the emulsion was diluted to 65% silicone solid content and 0.88 g of Protease B enzyme 42 mg/ml stock solution was added to this formulation. After each addition step, the sample was mixed two times in a Houschild AM-501 dental mixer. In the second formulation, 2% of the dry weight was replaced with glycerin and added in the same step as the PVA during the formulation. The prepared emulsions were spread on DC 7-4107 silicone membrane/Polycarbonate substrate using a draw down bar made by Paul N. Gardner Company, Inc. The drawn film was allowed to thy and cured to a thin film on the substrate over 24 hours in ventilated hood. From the dried film, patches were cut out and analyzed for enzyme release activity. The samples were tested using Franz Cell Assembly and N-succinyl-LAla-L-Ala-L-Pro-L-Phe-p-nitroanilide (suc-AAPF-pNA) assay for proteolytic activity. The Franz Cell body was filled with dissolution buffer (10 mM MES, 10 mM CaCl2, and 0.005% Tween 80 at pH 5.4) and a patch sample was attached on top of the cell. Samples from the cell were collected after 15 minutes, 1 hour, 2 hours, 4 hours, 8 hours and 24 hours. From each collected sample, aliquots were pipetted directly into a cuvette containing assay buffer (100 mM Tris and 0.005% Tween 80 at pH 8.6) and suc-AAPFpNA substrate. Then, the enzyme activity was measured on a UV/Visible spectrometer, which gave the concentration of enzyme in the dissolution buffer in mg/ml. FIG. 5 illustrates that glycerin enhances the rate of release of the Protease B enzyme from these matrices.

### Example 5

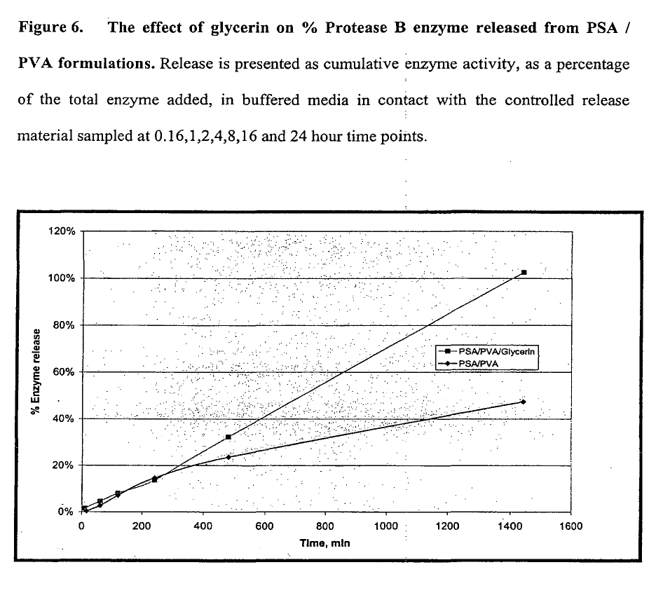

This experiment was conducted to evaluate the effect of glycerin on the sustained release of Protease B enzyme from a Dow Corning® PSA 7-4602 a pressure sensitive adhesive silicone matrix. First, the hydrophilic phase was prepared by mixing 8.71 g of hydrophilic carrier PVA solution (40% Mowiol 3-83) with 0.767 ml of Protease B enzyme 42 mg/nil stock solution. Then, 20.43 g of silicone phase was added to this mixture. After each addition step, the sample was mixed two times in a Houschild AM-501 dental mixer. In the second formulation, 2% of the dry weight was replaced with glycerin and added in the same step as the PVA during the formulation. The prepared emulsions were spread on DC 7-4107 silicone membrane/Polycarbonate substrate using a thaw down bar made by Paul N. Gardner Company, Inc. The drawn film was allowed to dry to a thin film on the substrate over 24 hours in a ventilated hood. From the dried film, patches were cut out and analyzed for enzyme release activity. The samples were tested using Franz Cell Assembly and N-succinyl-L-Ala-L-Ala-L-Pro-L-Phe-p-nitroanilide (suc-AAPF-pNA) assay for proteolytic activity. The Franz Cell body was filled with dissolution buffer (10 mM MES, 10 mM CaCl2, and 0.005% Tween 80 at pH 5.4) and a patch sample was attached on top of the cell. Samples from the cell were collected after 15 minutes, 1 hour, 2 hours, 4 hours, 8 hours and 24 hours. From each collected sample, aliquots were pipetted directly into a cuvette containing assay buffer (100 mM Tris and 0.005% Tween 80 at pH 8.6) and suc-AAPF-pNA substrate. Then, the enzyme activity was measured on a UV/Visible spectrometer, which gave the concentration of enzyme in the dissolution buffer in mg/ml. FIG. 6 illustrates that glycerin enhances the rate of release of Protease B enzyme from these matrices.

### Example 6

This experiment was conducted to evaluate the processing effect on the sustained release of LG-12 protease from a Dow Corning® PSA 7-4602 a pressure sensitive adhesive silicone matrix. First, the hydrophilic phase was prepared by mixing 13 g of hydrophilic carrier PVA solution (40% Mowiol 3-83) with 0.896 LG-12 protease enzyme stock solution. Then, 30 g of silicone phase was added to this mixture. In the first case, the silicone component was added in one step and, in the second case, the silicone was added in two steps. After each addition step, the sample was mixed two times in a Houschild AM-501 dental mixer. The prepared emulsions were spread on DC 7-4107 silicone membrane/Polycarbonate substrate using a draw down bar made by Paul N. Gardner Company, Inc. The drawn film was allowed to thy to a thin film on the substrate over 24 hours in a ventilated hood. From the dried film, patches were cut out and analyzed for enzyme release activity. The samples were tested using Hanson SR8 Plus Dissolution Tester and N-succinyl-L-Ala-L-Ala-L-Pro-L-Phe-p-nitroanilide (suc-AAPF-pNA) assay for proteolytic activity. The Hanson SR8 Plus Dissolution tester was filled with dissolution buffer (10 mM MES, 10 mM CaCl2, and 0.005% Tween 80 at pH 5.4) and a patch sample was placed inside of the vessel. Samples from the cell were collected after 10 minutes, 1 hour, 2 hours, 4 hours, 8 hours, 16 hours and 24 hours. From each collected sample, aliquots were pipetted directly into a cuvette containing assay buffer (100 mM Tris and 0.005% Tween 80 at pH 8.6) and suc-AAPF-pNA substrate. Then, the enzyme activity was measured on a UV/Visible spectrometer, which gave the concentration of enzyme in the dissolution buffer in mg/ml.

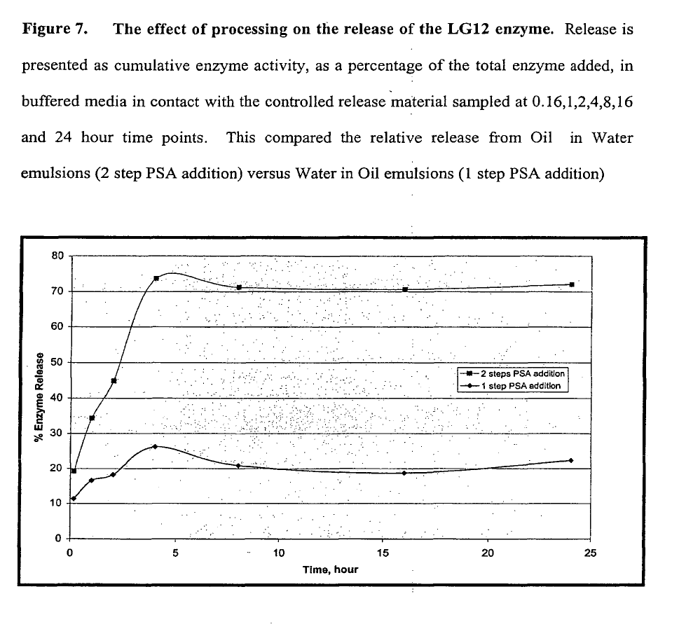

After examining the two types of samples in emulsion and film form, demonstrated differences were observed depending on whether the PSA silicone component was added in one or two steps to the hydrophilic phase. When the 7-4602 PSA was added in one step, the emulsion was thin and easy to spread on the 7-4107 membrane, while the dried film demonstrated good adhesion properties on the membrane. Less enzyme was released from this formulation as compared to the emulsion prepared using two steps. In contrast, when the 7-4602 was added in two steps, the resulting emulsion was thick and harder to spread on the 7-4107 membrane, while the dried film only adhered to the membrane with the aid of a 7-4600 PSA layer between the membrane and the film. The enzyme release from this formulation was greater than the enzyme release obtained from the film made using the one step process. FIG. 7 illustrates the results of the enzyme release from these matrices. Since the enzymatic release appeared to be dependent upon the addition steps in the formulation, a small amount of sample from each emulsion was mixed into water to test the nature of the emulsions. This investigation showed that when the 7-4602 PSA was added in one step, the emulsion did not combine with water. Comparatively, when 7-4602 PSA was added in two addition steps, the resulting emulsion dissipated into water. These results demonstrated that the first emulsion type was a W/O emulsion and the other sample was an 0/W emulsion, respectively. Since an increased % LG12 protease was observed to release from an O/W emulsion, the processing was determined to have an effect on the resultant type of emulsion.

### Example 7

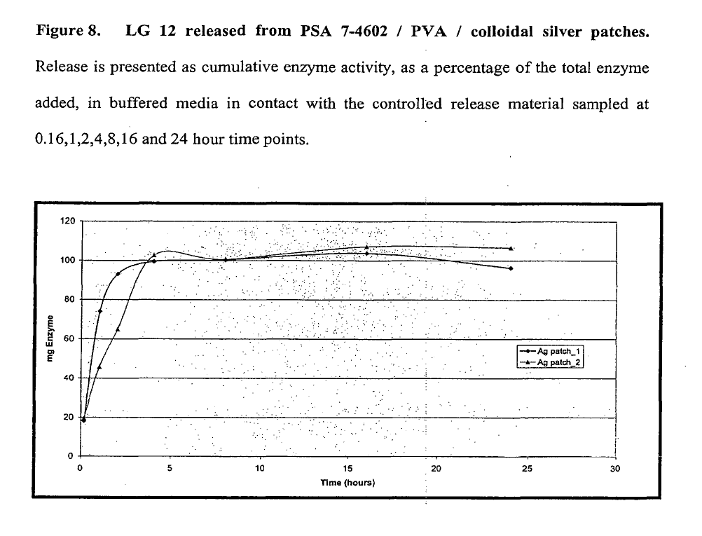

This experiment was conducted to evaluate the effect of polloidal silver on the sustained release of LG-12 protease from a Dow Corning® PSA 7-4602 a pressure sensitive adhesive silicone matrix. The 40.98 mg/ml enzyme stock solution was diluted 10 fold in the colloidal silver solution (Colloidal Silver from Natural Immunogenics Corp. Miami, Fla.). First, the hydrophilic phase was prepared by mixing 6.5 g of hydrophilic carrier PVA solution (40% Mowiol 3-83) with 1.596 ml LG-12 protease enzyme/colloidal silver solution. Then, 15 g of silicone phase was added to this mixture. After each addition step, the sample was mixed two times in a Houschild AM-501 dental mixer. The prepared emulsions were spread on DC 7-4107 silicone membrane! Polycarbonate substrate using a draw down bar made by Paul N. Gardner Company, Inc. The drawn film was allowed to dry to a thin film on the substrate over 24 hours in a ventilated hood. From the dried film, patches were cut out and analyzed for enzyme release activity. The samples were tested using Hanson SR8 Plus Dissolution Tester and N-succinyl-L-Ala-L-Ala-L-Pro-L-Phe-p-nitroanilide (suc-AAPF-pNA) assay for proteolytic activity. The Hanson SR8 Plus Dissolution tester was filled with dissolution buffer (10 mM MES, 10 mM CaCl2, and 0.005% Tween 80 at pH 5.4) and a patch sample was placed inside of the vessel. Samples from the cell were collected after 10 minutes, 1 hour, 2 hours, 4 hours, 8 hours, 16 hours and 24 hours. From each collected sample, aliquots were pipetted directly into a cuvette containing assay buffer (100 mM Tris and 0.005% Tween 80 at pH 8.6) and suc-AAPF-pNA substrate. Then, the enzyme activity was measured on a UV/Visible spectrometer, which gave the concentration of enzyme in the dissolution buffer in mg/ml. FIG. 8 illustrates that the release of the LGI2 protease from this matrix was complete after 4 hours.

In addition, the enzyme release was also followed on a skim milk plate.

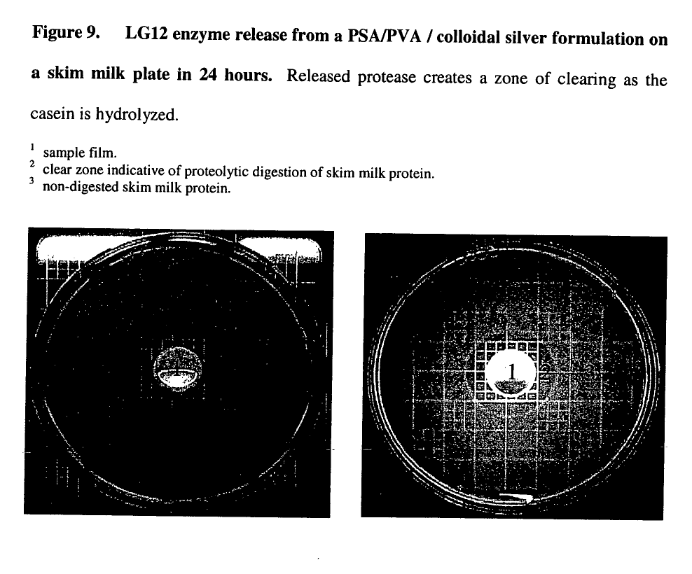

Skim milk plate preparation: In one bottle, skim milk powder was dissolved in water, and, in an other bottle, yeast extract, sodium chloride, and agar were mixed together. The bottles were autoclaved at 121° C. for 15 minutes using Hirayama Autoclave. Then, they were allowed to cool to 45° C. in a water bath. Afterward, the skim milk solution was added to the agar and the combined solution was poured into petri dishes and allowed to solidify. A small piece of the silicone matrix film containing the enzyme was cut out and pressed into the skim milk agar. The petri dish plate was allowed to incubate at 30±2° C. FIG. 9 shows the enzyme released from the prepared patch on a skim milk plate after 24 hours incubation.

### Example 8

This experiment was conducted to evaluate the effect of DC 5700 silane on the sustained release of LG-12 protease from a Dow Corning® PSA 7-4602 a pressure sensitive adhesive silicone matrix. First, the hydrophilic phase was prepared by mixing 6.54 g of hydrophilic carrier PVA solution (40% Mowiol 3-83) with 0.767 ml of Protease B enzyme 42 mg/ml stock solution. Then, 2.17 g of DC 5700 silane followed by 20.43 g of silicone phase was added to this mixture. After each addition step, the sample was mixed two times in a Houschild AM-501 dental mixer. The prepared emulsions were spread on DC 7-4107 silicone membrane! Polycarbonate substrate using a draw down bar made by Paul N. Gardner Company, Inc. The drawn film was allowed to dry to a thin film on the substrate over 24 hours in a ventilated hood. From the dried film, patches were cut out and analyzed for enzyme release activity. The enzyme release was followed on a skim milk plate.

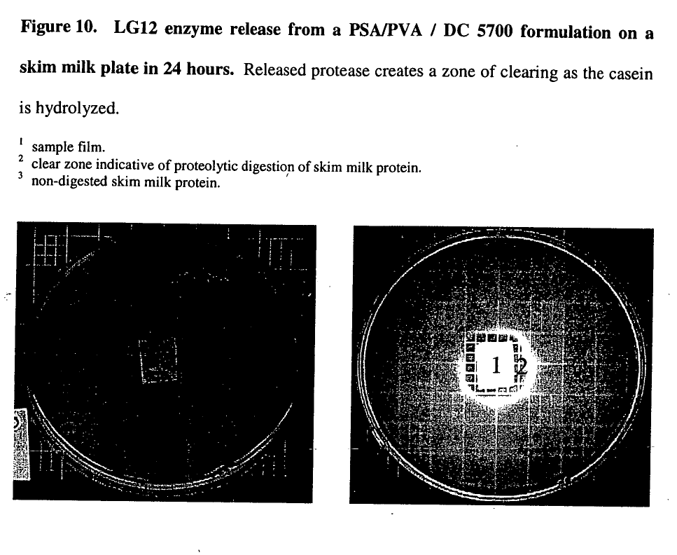

Skim milk plate preparation: In one bottle, skim milk powder was dissolved in water, and, in an other bottle, yeast extract sodium chloride, and agar were mixed together. The bottles were autoclaved at 121° C. for 15 minutes using Hirayama Autoclave. Then they were allowed to cool down to 45° C. in a water bath. Afterward, the skim milk solution was added to the agar and the combined solution was poured into petri dishes and allowed to solidify. A small piece of the silicone matrix film containing the enzyme was cut out and pressed into the skim milk agar. The petri dish plate was allowed to incubate at 30±2° C. FIG. 10 shows the enzyme release from the prepared patch on a skim milk plate after 24 hours incubation. A small amount of enzyme was observed to release from this formulation.

### Example 9

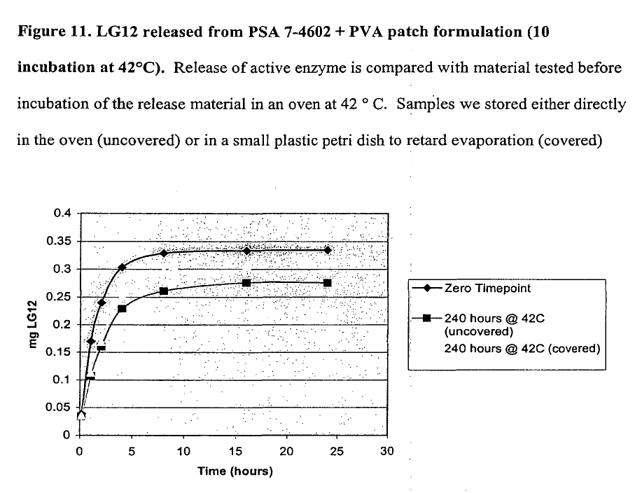

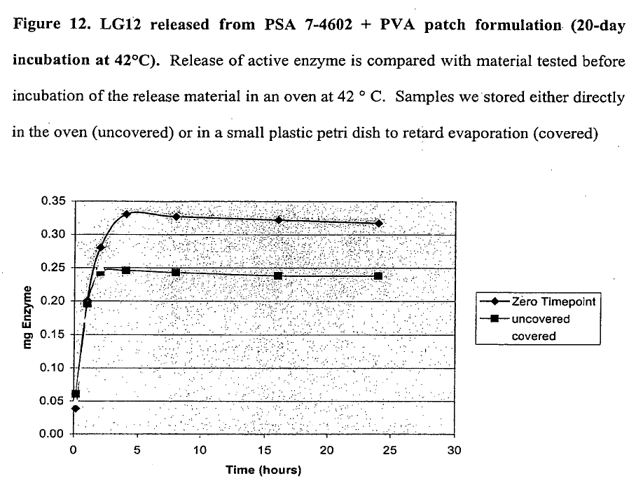

This experiment was conducted to evaluate enzyme stability in the silicone matrix. Dried PSA 7-4602 silicone+PVA films containing approximately 0.400 mg LGI2 per gram dry weight were cut into 3.5 cm discs and incubated in a SYBRON Thermolyne Type 3700 culture incubator set at 42° C. for 10 days and 20 days. Half of the of the discs were placed inside an autoclave pouch to minimize moisture loss whereas the other half were left uncovered. Unincubated discs were weighed and loaded into a dissolution vessel of a Hanson SR8 Plus Dissolution Tester and used as control for this experiment. The dissolution buffer used was a 10 mM MES pH 5.2+0.005% Tween-80+10 mM calcium chloride. Fractions were collected after 10 minutes, 1, 2, 4, 8, 16 and 24 hours. These fractions were assayed for protease activity using N-succinyl-L-Ala-L-Ala-L-Pro-L-Phe-p-nitroanilide as the substrate to quantitate the amount of enzyme released from the silicone discs. As for the 42° C.-incubated discs, the amount of remaining active enzyme released from the discs were measured using the same assay. FIGS. 11 and 12 illustrate the effect of moisture loss on enzyme stability in the silicone matrix.

### Example 10

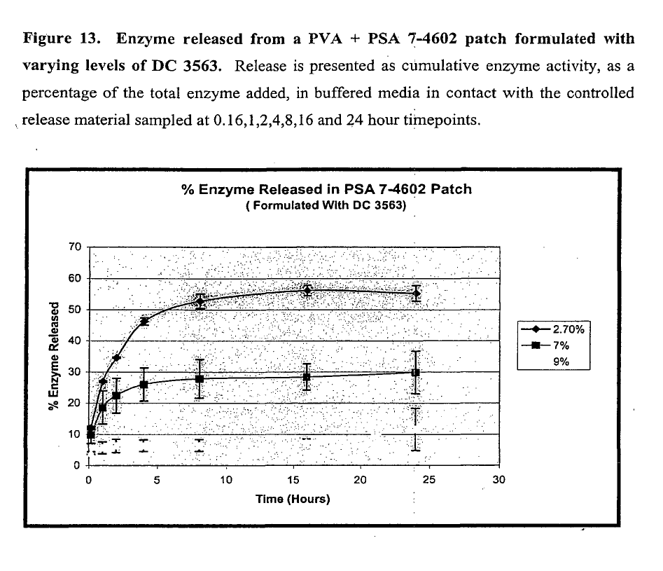

This experiment was conducted to create a more tacky silicone+PVA+enzyme film with the addition of DC 3563. Varying levels of DC 3563 were mixed with Dow Corning® PSA 7-4602, the silicone phase. This silicone mixture was then added to the hydrophilic phase which contains both the PVA solution (40% Mowial 3-83 in water) and the Protease B enzyme to make an emulsion. The emulsions were spread into films on a DC 7-4 107 attached to a Mylar substrate using a metal spreader made by Paul N. Gardner Company, Inc. Dried films were cut into 3.5 cm diameter discs, weighed and loaded into a dissolution vessel of a Hanson SR8 Plus Dissolution Tester. The dissolution buffer used was a 10 mM MES pH 5.2+0.005% Tween-80+10 mM calcium chloride. Fractions were collected after 10 minutes, 1, 2, 4, 8, 16 and 24 hours. These fractions were assayed for protease activity using N-succinyl-L-Ma-L-Ala-L-Pro-L-Phe-p-nitroanilide as the substrate to quantitate the amount of enzyme released from the silicone discs. FIG. 13 illustrates the effect of varying levels of DC 3563 on enzyme release.

### Example 11

This experiment illustrates the superiority of enzyme efficacy in wound debridement when released from one embodiment of the controlled release system over enzyme released from an ointment and enzyme released from a known commercial debridement cream.

The dressing embodiment according to the present invention comprises three layers. An outer layer of silicone elastomeric membrane (DCC 4107) is in contact with a pressure sensitive adhesive (PSA) (DCC 4602). The PSA extends over the entire dressing, which is in the form of squares cut to a dimension depending on the size of the controlled release composition layer (in the form of an enzyme-releasing silicone patch). A disc of the CR layer is placed on the inner surface and is formulated to release a specific but variable dosage of the CR composition comprising Genencor protease LG12 or FNA release emulsions. The protease-release emulsion is characterized as an oil-in-water emulsion. Previous studies demonstrate that under conditions of total immersion this material releases 100% of the protease over an 8 hour period and the enzyme is reported to retain 85-88% of its activity in accelerated stability studies intended to represent 18-month storage at room temperature.

Studies are conducted using a pig burn eschar as a model for in vivo efficacy of the enzyme. Pig derma is considered to be the most similar non-primate skin to human skin. Release of the enzyme from Controlled release layers and K-Y Jelly®, each comprising three concentrations (1×, 10× and 100×) of protease, were compared. In addition, emulsion without protease, KY-Jelly without protease, no treatment, and Accuzyme® were similarly tested as representative of existing debridement creams. Burn wounds were introduced in a 3×5 array on each side of an adolescent pig and each side was selected randomly to be the silicone dressing or ointment side. The ointment side was covered with a secondary dressing to prevent the pig from rubbing the dressing material.

After 24 hours, a dose response (response is removal of eschar from the surface of the wound as visually observed) was observed visually after the first treatment with the silicone release dressing. Some inflammation, evidenced by redness, was noted around the edges of the 100× treatment. There was no clear dose response after 24 hours in the KY treatments. The commercially available debridement product, Accuzyme®, yielded results similar to the KY Jelly.

After 48 hours similar dose responses are noted. The 100× patch showed increased removal of the burn eschar over both the 10× patch after 48 hours, and the 100× after 24 hours, indicating that there may be an even more desirable duration of dose. In some wound regions there is 100% debridement after 48 hours. After 48 hours there is very little discernable difference between the dose levels in the KY treatments.

### EXPERIMENTAL

1. Emulsion Preparation

The “1×” patches were formulated by first mixing 13.01 g 40% Mowiol PVA (3-83) in deionized water and 3.41 g of a solution of LG12 (0.3904 mg/ml). The resulting mixture was then mixed with 15.68 g PSA (59% DC 7-4602 in Ethyl Acetate) followed by an additional 14.24 g PSA followed by another mixing step.

The “10×” patches were formulated by first mixing 13 g 40% Mowiol PVA (3-83) in deionized water and 3.40 g of a solution of LG12 (3.904 mg/ml). The resulting mixture was then mixed with 16.40 g PSA (59% DC 7-4602 in Ethyl Acetate) followed by an additional 13.31 g PSA followed by another mixing step.

The “100×” patches were formulated by first mixing 13.01 g 40% Mowiol PVA (3-83) in deionized water and 3.42 g of a solution of LG12 (39.04 mg/ml). The resulting mixture was then mixed with 16.52 g PSA (59% DC 7-4602 in Ethyl Acetate) followed by an additional 13.65 g PSA followed by another mixing step. All mixing steps were performed in a dental mixer (Houschild AM-501). Each mixing step was repeated twice for 22 seconds.

2. Dressing Manufacture

Emulsions are spread on DC 7-4107 silicone membrane/polycarbonate substrate using a draw down bar made by Paul N. Gardner Company, Inc. to form a controlled release layer according to one embodiment of the present invention. The drawn film is allowed to dry to a thin film on the substrate over 24 hours in a ventilated hood. From the dried film 1.5 cm. diameter patches were punched out.

The adhesive layer is prepared by coating 7-4602 PSA on Fluoro release liner (Scotch Pack 9956). The release liner with 8 mil shims are laid down and clamped to the draw down table. A bar is positioned across the holders and squeezed down with weight. Adequate amounts of 7-4602 silicone adhesive is poured next to the bar and subsequently the motor is activated to draw a fine continuous film. The film is allowed to dry overnight. 7-4107 silicone membrane/polycarbonate substrate is laminated over the dried film and this is cut into 2.5 cm2 squares. The 7-4600 adheres to the membrane and the Fluoro release liner can be peeled off.

The 1.5 cm. diameter controlled release discs are adhered to the centers of the 7-4600 PSA layer of the square laminates.

### Example 12

This example illustrates the achievement of nearly 100% removal of burn eschar after 48 hours of treatment with a multiple layer dressing comprising a silicon release emulsion of LG12 protease according to the present invention. It further illustrates that the inventive multiple layer dressings do not cause irritation to healthy skin indicating that observed irritation of healthy skin surrounding a treated burn is due to wound exudate and not the dressing, per se.

In particular, experiments are conducted to explore increasing dosage benefits (250× and 600×), altered treatment strategies (1×48 or 2×24 hour regimens), and safety (irritation of healthy skin). The study demonstrates that a longer duration of treatment (1×48 hours) permitted more moisture build-up which assures complete release of protease and an increase in moisture to the wound. These results were compared to 2×24 hour treatment regimens. Safety was studied by increasing the dimension of the silicone/protease releasing emulsion to overlap healthy tissue and by applying a separate patch on healthy tissue. Both of these followed the 48 and 2×24 hour treatment regimens. The protease used for these studies is LG12.

The debridement was observed to be more extensive at the 250× and 600× levels resulting in bowl-like depressions where burn eschar had been. There is extensive redness and irritation in the adjoining healthy tissue. This irritation is attributable to wound exudates which comprise increased levels of bacterial endotoxins and more serum leakage. Similar results are seen for the same dosages given once and left on for 48 hours. The most extensive irritation was observed at the 600× dosage level in the 2×24 hour treatment regimen. Histology sections of the wounds showed that, after 48 hours of treatment, 50-90% of the eschar is removed for the 100× dose, 70-90% for the 250× dose, and 100% for the 600× dose.

After the 48 hours the patches were retrieved, stored frozen, and tested for detectable protease in a start modified immersion bath. All of the patches placed over wounds had little detectable protease. Patches placed on healthy tissue for 24 hours retained 90-99% activity and patches that had been placed on healthy tissue for 48 hours exhibited enzyme activity retention ranging from 70-95%. Healthy skin exhibited no irritation or tissue damage at any dose. It is considered that this could be partially due to a lack of sufficient moisture on the healthy skin necessary to trigger enzyme release. However, even a 15% release at the 600× level is the equivalent exposure to the 100× dose over 48 hours and no irritation was observed.

### EXPERIMENTAL

1. Emulsion Preparation

The “100×LG12” patches were formulated by first mixing 13.05 g 40% Mowiol PVA (3-83) in deionized water, 5.79 g of a solution of LG12 (23 mg/ml) and 0.73 g buffer (40% Propylene glycol, 25 mM Sodium formate, 5 mM Calcium chloride). The resulting mixture was then mixed with 15.77 g PSA (59% DC 7-4602 in Ethyl Acetate) followed by an additional 14.12 g PSA followed by another mixing step.

The “250×LG12” patches were formulated by first mixing 13.0 g 40% Mowiol PVA (3-83) in deionized water, 5.44 g of a solution of LG12 (61.5 mg/ml) and 1.13 g buffer (40% Propylene glycol, 25 mM Sodium formate, 5 mM Calcium chloride). The resulting mixture was then mixed with 15.03 g PSA (59% DC 7-4602 in Ethyl Acetate) followed by an additional 14.73 g PSA followed by another mixing step.)

The “600×LG12” patches were formulated by first mixing 12.98 g 40% Mowiol PVA (3-83) in deionized water and 6.6 g of a solution of LG12 (123 mg/ml). The resulting mixture was then mixed with 15.04 g PSA (59% DC 7-4602 in Ethyl Acetate) followed by an additional 14.85 g PSA followed by another mixing step.

The “100× FNA” patches were formulated by first mixing 13.01 g 40% Mowiol PVA (3-83) in deionized water, 5.68 g of a solution of FNA (24 mg/ml) and 0.96 g buffer (40% Propylene glycol, 25 mM Sodium formate, 5 mM Calcium chloride). The resulting mixture was then mixed with 14.84 g PSA (59% DC 7-4602 in Ethyl Acetate) followed by an additional 15.18 g PSA followed by another mixing step.

All mixing steps were performed in a dental mixer (Houschild AM-501). Each mixing step was repeated twice for 22 seconds.

2. Dressing Manufacture

The prepared emulsions were spread on DC 7-4107 silicone membrane/Polycarbonate substrate using a draw down bar made by Paul N. Gardner Company, Inc. The drawn film was allowed to dry to a thin film on the substrate over 24 hours in a ventilated hood. From the dried film 2.2 cm diameter patches were punched out.

The adhesive backing prepared by coating the 7-4602 PSA on Fluoro release liner (Scotch Pack 9956) following procedure outlined here. The release liner with 8 mil shims were laid down and clamped to the draw down table. A bar was positioned across the holders and squeezed down with weight. Adequate amount of 7-4602 silicone adhesive was poured next to the bar and subsequently the motor was activated to draw a fine continuous film. This film was allowed to dry overnight. 7-4107 silicone membrane/Polycarbonate substrate laminated over the dried film and it was cut to a dimension of 3.5×3.5 cm² squares. The 7-4600 adheres to the membrane and the Fluoro release liner can be peeled off.

The 2.2 cm diameter dried enzyme containing emulsion film discs were adhered to the centers of the 7-4600 PSA layer of the square laminates.

3. Enzyme Release Activity

Hanson SR8 Plus™ Dissolution Tester was used for the dissolution of the discs/patches to monitor sustained release of the protease enzyme from the silicone discs for a 24 hour time period. Paddles were attached for automated mixing and they were adjusted such that they were at a uniform distance from the silicone patches. Patches were loaded into designated wells of the 150 mL Hanson dissolution vessels with the silicone patch facing up. These vessels were then placed into the dissolution test station bath set at 31° C. temperature. Pre-equilibrated 10 mM MES pH 5.2 buffer with 0.005% Tween-80 and 10 mM calcium chloride were added into the vessel, preferably in a volume of 25 mL. The dissolution program was started using standardized parameters such as 50 rpm agitation, water bath temperature controlled at 31° C., and fixed collection times. The program was modified if needed. A total of 42 glass tubes were placed in a tube rack in the Auto Plus MultiFill™ fraction collector part of the machine. These were covered with saran wrap to avoid moisture loss as well as to filter dust particles.

The Hanson SR8 Plus™ Dissolution Tester was also equipped with an Auto Plus MultiFill™ fraction collector that collects sample aliquots after 10 minutes, 1 hour, 2, 4, 8, 16, and 24 hour time points. The aliquots were then assayed for protease activity using the N-succinyl-L-Ala-L-Ala-L-Pro-L-Phe-p-nitroanilide substrate. Then, the enzyme activity was measured on an UV/Visible spectrometer, which gave the concentration of enzyme in the dissolution buffer in mg/ml. The assay results of the first and second animal trial samples are presented in FIG. 6 and FIG. 11, respectively.

4. Maximum Dosage Determination

In the pig from example 11, dressings were prepared with a standard formula that gave the “100×” dose, which was arbitrarily targeted to be 5.0 mg enzyme/g dried emulsion. This involved mixing approximately 7% by weight of an enzyme solution with 21% PVA and the remainder PSA DC4602. The dosage is a function of the concentration and volume of enzyme solution used for the formulation. Thus two approaches can be employed to increase the enzyme concentration of the emulsion: increase the volume of enzyme solution added and/or increase the concentration of the protein solution used. Both approaches are bounded—the first one by when the nature of the emulsion is compromised by too high a dilution arising from increasing the enzyme solution used in formulation and the second one limited by the solubility of the enzyme. To determine the present limits, the emulsion was formulated and compared by adding 1×, 2×, 3× the amount of enzyme buffer solution without further changes in the recipe. The emulsion's consistency and the thickness of the dried emulsion was unchanged with the addition of 2× the volume of buffer solution but when 3× was added, the dried emulsion was thinner and resulted in lower weight patches, thus the net load was dispersed over a larger area resulting in a lower release/unit area, which is not desired. However, it was also possible to increase the concentration of the enzyme solution used 3× so that in combination with the benefit gains by mixing 2× the volume of enzyme solution, we were be able to achieve a 6-fold increase of the dosage.

The pig had 20 burn wounds, 2×5 on each side of the pig. To achieve complete wounding, the wounds were achieved as three 20 second burns. The diameter is increased from 1.5 to 2.2 and the final dressing square is 3.5 cm2 rather than 2.5 cm2 as disclosed in Example 11. Given that the amount of enzyme loaded could be increased by 6-fold it was dosed at the highest level previously tested 100×, at an intermediate of the maximum 250× and at the maximum level 600× corresponding to 5.0, 12.5 and 30 mg enzyme/g of dried emulsion. Since larger patches of (2.2 vs 1.5 cm) emulsion were being used, this would correspond to an increasing the amount of enzyme released by an additional factor of 2.1 arising from the increase in the patch size. Thus the consequence of approximately 200×, 500× and 1200× the amount of enzyme applied to wounds on the animal from Example 11 was being examined.

### Example 14

This example illustrates a method for achieving full wound debridement within a 24 hour treatment period with substantial elimination of skin irritation due to prolonged contact with wound exudates. Dressings comprising both high and low doses of the controlled release compositions exhibited a dose responsive effectiveness of the dressing with regard to debridement of burn eschar and the high dose achieved complete debridement within a 24 hour period.

Preparation of the Multi-Layer Dressings:

“2.1 mg LG12” patches are formulated by first mixing 3.25 g of 40% Mowiol PVA (3-83) in deionized water with 0.646 ml of a stock solution of LG12 (123 mg/ml in buffer) and 0.9 ml of buffer (40% Propylene glycol, 25 mM Sodium formate, 5 mM Calcium chloride). The resulting mixture is then mixed with 3.67 g of PSA (59% DC 7-4602 in Ethyl Acetate) followed by an additional 3.88 g of PSA and followed by another mixing step.

“4.9 mg LG12” patches are formulated by first mixing 3.26 g of 40% Mowiol PVA (3-83) in deionized water with 1.546 ml of a solution of LG12 (127 mg/ml). The resulting mixture is then mixed with 3.86 g of PSA (59% DC 7-4602 in Ethyl Acetate) followed by an additional 3.67 g of PSA and followed by another mixing step.

All mixing steps are performed in a dental mixer (Houschild AM-501). Each mixing step is repeated twice for 22 seconds.

The prepared emulsions are spread on DC 7-4107 silicone membrane/Polycarbonate substrate using a draw down bar made by Paul N. Gardner Company, Inc. The drawn film is allowed to dry to a thin film on the substrate over 72 hours in a ventilated hood. (1)

The adhesive layer is prepared by coating the 7-4602 PSA on Fluoro release liner (Scotch Pack 9956) following procedure outlined here. The release liner with 8 mil shims are laid down and clamped to the draw down table. A bar is positioned across the holders and squeezed down with weight. Adequate amount of 7-4602 silicone adhesive is poured next to the bar and subsequently the motor is activated to draw a fine continuous film. This film is allowed to dry overnight. 7-4107 silicone membrane/Polycarbonate substrate laminated over the dried film. The 7-4600 adheres to the membrane and the Fluoro release liner can be peeled off.

The dried emulsion is laminated over the adhesive/membrane composition and 2.2 cm diameter patches are punched out of this multi-layer design.

The multi-layer controlled release design is then adhered to the Mepitel® silicone mesh. An adsorbent layer is adhered to the other side of the silicone mesh to adsorb the exudate during the debridement process.

An embodiment of the multi-layer patch employed in this example is illustrated by FIG. 14A. The relative effectiveness in simultaneous management of wound exudate and debridement is illustrated by comparative FIG. 15. FIG. 15A illustrates results achieved by employment of a multi-layer dressing having the absorbent layer in direct contact with the out-lying skin area. FIG. 15B is the result achieved by employment of a multi-layer dressing comprising a combination porous adhesive silicone mesh and absorbent layer topped by an occlusive backing (Mepilex®border), and FIG. 15C is the result achieved by employment of a combination porous adhesive silicone mesh (Mepitel®) and an absorbent layer, which is adhered to the other side of the silicone mesh to adsorb the exudate during the debridement process. In the latter two embodiments, the exudate absorbing layer is separated from the skin by the silicone mesh adhesive, and the absorbent layer is further kept separate from the controlled-release layer by an intervening occlusive layer.

What is claimed is:

1. A multi-layer dressing for topical application to a substrate, said dressing comprising:
(A) a controlled-release layer formed from a controlled-release composition comprising;
(i) an oil-in-water or water-in-oil emulsion, and
(ii) an active agent incorporated into said emulsion and comprising a protein;
(B) an adhesive layer disposed adjacent said controlled-release layer; and
(C) an additional layer selected from the group of a backing layer, a cushioning layer, an absorbent layer, a second adhesive layer, and combinations thereof,
wherein the adhesive layer comprises a silicone adhesive, a non-silicone adhesive, or combinations thereof.

2. The multilayer dressing according to claim 1, wherein the non-silicone adhesive is selected from the group consisting of acrylic adhesive, rubber adhesive, and combinations thereof.

3. The multilayer dressing according to claim 2, wherein the non-silicone adhesive comprises an acrylic adhesive.

4. The multilayer dressing according to claim 3 wherein the acrylic adhesive comprises acrylic polymers and an aqueous or nonaqueous solvent.

5. The multilayer dressing according to claim 4, wherein the acrylic adhesive comprises acrylic polymers and an aqueous solvent.

6. The multilayer dressing according to claim 5, wherein the acrylic adhesive is selected from the group consisting of DC®MG-0560, and DC® MG-0580.

7. The multilayer dressing according to claim 4, wherein the acrylic adhesive is selected from the group consisting of DC® MG-0610 and DC® MG-0607.

8. The multilayer dressing according to claim 2, wherein the non-silicone adhesive comprises a rubber adhesive, said rubber adhesive comprising a styrene-butadiene copolymer.

9. The multilayer dressing according to claim 8, wherein the rubber adhesive comprises DC® MG-0156.

10. The multilayer dressing according to claim 1 comprising a patch.

11. A multi-layer dressing for topical application to a substrate, said dressing comprising:
(A) a controlled-release layer formed from a controlled-release composition comprising:
(i) an oil-in-water or water-in-oil emulsion, and
(ii) an active agent incorporated into said emulsion and comprising a protein; and
(B) an adhesive layer disposed adjacent said controlled-release layer;
(C) an occlusive layer disposed adjacent said adhesive layer and away from said controlled release layer;
(E) an absorbent layer disposed opposite the occlusive layer from the controlled release layer; and,
(F) optionally, at least one additional layer selected from the group of a backing layer, a cushioning layer, a second absorbent layer, a second adhesive layer, a porous adhesive layer, and combinations thereof.

12. The multilayer dressing according to claim 11, wherein an additional layer comprises a porous adhesive layer disposed adjacent the absorbent layer and between the absorbent layer and the occlusive layer.

13. The multilayer dressing according to claim 12, wherein the porous adhesive layer comprises a silicone mesh.

14. The multilayer dressing according to claim 12, wherein the porous adhesive layer comprises Mepitel® silicone mesh.

15. The multilayer dressing according to claim 12, wherein the controlled release layer, the adhesive layer, and the occlusive layer are all substantially co-extensive in area, the area equal to C.

16. The multilayer dressing according to claim 15, wherein the porous adhesive layer and the absorbent layer are substantially co-extensive in area, the area equal to A.

17. The multilayer dressing according to claim 16, wherein the area A is greater than the area C and extends beyond C to form a border area, B, further wherein the porous adhesive layer contacts and adheres the dressing to the substrate.

18. A method of managing wound exudates, the method comprising: adhering a multilayer dressing to a substrate for an effective amount of time, the multilayer dressing comprising:
(A) a controlled-release layer formed from a controlled-release composition comprising:
(i) an oil-in-water or water-in-oil emulsion, and
(ii) an active agent incorporated into said emulsion and comprising a protein; and
(B) an adhesive layer disposed adjacent said controlled-release layer;
(C) an occlusive layer disposed adjacent said adhesive layer and away from said controlled release layer;
(E) an absorbent layer disposed opposite the occlusive layer from the controlled release layer; and,
(F) optionally, at least one additional layer selected from the group of a backing layer, a cushioning layer, a second absorbent layer, a second adhesive layer, a porous adhesive layer, and combinations thereof.

19. The method of managing wound exudates according to claim 18, wherein the occlusive layer directs the controlled release composition toward the wound.

20. The method of managing wound exudates according to claim 18, wherein the porous silicone adhesive layer directs wound exudate toward the absorbent layer.

21. The method of managing wound exudates according to claim 18, wherein the substrate comprises skin.

22. The method of managing wound exudates according to claim 18, wherein the at least one additional layer comprises a porous adhesive layer disposed adjacent the absorbent layer and between the absorbent layer and the occlusive layer.

23. The method of managing wound exudates according to claim 18, wherein the porous adhesive layer comprises a silicone mesh.

24. The method of managing wound exudates according to claim 23, wherein the porous adhesive layer comprises Mepitel® silicone mesh.

25. The method of managing wound exudates according to claim 22, wherein the controlled release layer, the adhesive layer, and the occlusive layer are all substantially co-extensive in area, the area equal to C.

26. The method of managing wound exudates according to claim 25, wherein the porous adhesive layer and the absorbent layer are substantially co-extensive in area, the area equal to A.

27. The method of managing wound exudates according to claim 26, wherein the area A is greater than the area C and extends beyond C to form a border area, B, further wherein the porous adhesive layer contacts and adheres the dressing to the substrate.

28. A method of achieving noninvasive debridement of wound eschar, the method comprising: adhering a multilayer dressing to a substrate, wherein the multilayer dressing comprises:
(A) a controlled-release layer formed from a controlled-release composition comprising:
(i) an oil-in-water or water-in-oil emulsion, and
(ii) an active agent incorporated into said emulsion and comprising a protein; and
(B) an adhesive layer disposed adjacent said controlled-release layer;
(C) an occlusive layer disposed adjacent said adhesive layer and away from said controlled release layer;
(E) an absorbent layer disposed opposite the occlusive layer from the controlled release layer; and,
(F) optionally, at least one additional layer selected from the group of a backing layer, a cushioning layer, a second absorbent layer, a second adhesive layer, a porous adhesive layer, and combinations thereof.

29. The method of achieving noninvasive debridement of wound eschar according to claim 28, wherein the occlusive layer directs the controlled release composition toward the wound.

30. The method of achieving noninvasive debridement of wound eschar according to claim 28, wherein the porous silicone adhesive layer directs wound exudate toward the absorbent layer.

31. The method of achieving noninvasive debridement of wound eschar according to claim 28, wherein the substrate comprises skin.

32. The method of achieving noninvasive debridement of wound eschar according to claim 28, wherein the at least one additional layer comprises a porous adhesive layer disposed adjacent the absorbent layer and between the absorbent layer and the occlusive layer.

33. The method of achieving noninvasive debridement of wound eschar according to claim 32, wherein the porous adhesive layer comprises a silicone mesh.

34. The method of achieving noninvasive debridement of wound eschar according to claim 32, wherein the porous adhesive layer comprises Mepitel® silicone mesh.

35. The method of achieving noninvasive debridement of wound eschar according to claim 32, wherein the controlled release layer, the adhesive layer, and the occlusive layer are all substantially co-extensive in area, the area equal to C.

36. The method of managing wound exudates according to claim 32, wherein the porous adhesive layer and the absorbent layer are substantially co-extensive in area, the area equal to A.

37. The method of achieving noninvasive debridement of wound eschar according to claim 36, wherein the area A is greater than the area C and extends beyond C to form a border area, B, further wherein the porous adhesive layer contacts and adheres the dressing to the substrate.

38. The method of achieving noninvasive debridement of wound eschar as set forth in claim 28, wherein the wound is a chronic wound.
